SEQUENCE LISTING (34 sequences)

SEQ ID NO: 1 - DNA, 58 bp, Artificial Sequence
  Description of Artificial Sequence Synthetic primer
ggcgcgcctaaccatctatttcaaggagacagtcataatgaagaagctcctctttgct

SEQ ID NO: 2 - DNA, 58 bp, Artificial Sequence
  Description of Artificial Sequence Synthetic primer
ggcgcgcctaaccatctatttcaaggagacagtcataatgaaaaagcttttattcatg

SEQ ID NO: 3 - DNA, 57 bp, Artificial Sequence
  Description of Artificial Sequence Synthetic primer
ggcgcgcctaaccatctatttcaaggaacagtcttaatgaaaaagcttttattcatg

SEQ ID NO: 4 - DNA, 21 bp, Artificial Sequence
  Description of Artificial Sequence Synthetic primer
ctgggctgcctggtcaaggac

SEQ ID NO: 5 - DNA, 20 bp, Artificial Sequence
  Description of Artificial Sequence Synthetic primer
cgcaattcctttagttgttc

SEQ ID NO: 6 - DNA, 64 bp, Artificial Sequence
  Description of Artificial Sequence Synthetic primer
agcttcaacaggggagagtgttaataaggcgcgcctaaccatctatttcaaggaacagtcttaa

SEQ ID NO: 7 - DNA, 66 bp, Artificial Sequence
  Description of Artificial Sequence Synthetic primer
cagtggcccctacagaatgttcataataaggcgcgcctaaccatctatttcaaggagacagtcata

SEQ ID NO: 8 - DNA, 66 bp, Artificial Sequence
  Description of Artificial Sequence Synthetic primer
cagtggcccctgcagaatgctcttaataaggcgcgcctaaccatctatttcaaggagacagtcata

SEQ ID NO: 9 - DNA, 22 bp, Artificial Sequence
  Description of Artificial Sequence Synthetic primer
gttcctttctattctcacagtg

SEQ ID NO: 10 - DNA, 20 bp, Artificial Sequence
  Description of Artificial Sequence Synthetic primer
gcaccctcctccaagagcac

SEQ ID NO: 11 - DNA, 63 bp, Artificial Sequence
  Description of Artificial Sequence Synthetic primer
atgaagaagctcctctttgctatcccgctcgtcgttccttttgtggcccagccggccatggcc

SEQ ID NO: 12 - DNA, 42 bp, Artificial Sequence
  Description of Artificial Sequence Synthetic primer
taataaggcgcgcctaaccatctatttcaaggaacagtctta

SEQ ID NO: 13 - DNA, 33 bp, Artificial Sequence
  Description of Artificial Sequence Synthetic primer
cctttagttgttcctttctattctcacagtgca

SEQ ID NO: 14 - DNA, 25 bp, Artificial Sequence
  Description of Artificial Sequence Synthetic primer
ggaggagggtgctagcgggaagacc

SEQ ID NO: 15 - DNA, 21 bp, Artificial Sequence
  Description of Artificial Sequence Synthetic primer
atgaagaagctcctctttgct

SEQ ID NO: 16 - DNA, 50 bp, Artificial Sequence
  Description of Artificial Sequence Synthetic primer
ctaaccatctatttcaaggaacagtcttaatgaagaagctcctctttgct

SEQ ID NO: 17 - DNA, 49 bp, Artificial Sequence
  Description of Artificial Sequence Synthetic primer
ttgaaatagatggttaggcgcgccttattaacactctcccctgttgaag

SEQ ID NO: 18 - DNA, 24 bp, Artificial Sequence
  Description of Artificial Sequence Synthetic primer
acactctcccctgttgaagctctt

SEQ ID NO: 19 - DNA, 25 bp, Artificial Sequence
  Description of Artificial Sequence Synthetic primer
tgaacattctgtaggggctactgtc

SEQ ID NO: 20 - DNA, 25 bp, Artificial Sequence
  Description of Artificial Sequence Synthetic primer
tgaacattctgtaggggccactgtc

SEQ ID NO: 21 - DNA, 25 bp, Artificial Sequence
  Description of Artificial Sequence Synthetic primer
tgaacattccgtaggggcaactgtc

SEQ ID NO: 22 - DNA, 25 bp, Artificial Sequence
  Description of Artificial Sequence Synthetic primer
agagcattctgcaggggccactgtc

SEQ ID NO: 23 - DNA, 50 bp, Artificial Sequence
  Description of Artificial Sequence Synthetic primer
ttgaaatagatggttaggcgcgccttattatgaacattctgtaggggcta

SEQ ID NO: 24 - DNA, 49 bp, Artificial Sequence
  Description of Artificial Sequence Synthetic primer
ttgaaatagatggttaggcgcgccttattatgaacattctgtaggggcc

SEQ ID NO: 25 - DNA, 50 bp, Artificial Sequence
  Description of Artificial Sequence Synthetic primer
ttgaaatagatggttaggcgcgccttattatgaacattccgtaggggcaa

SEQ ID NO: 26 - DNA, 49 bp, Artificial Sequence
  Description of Artificial Sequence Synthetic primer
ttgaaatagatggttaggcgcgccttattaagagcattctgcaggggcc

SEQ ID NO: 27 - DNA, 9473 bp, Artificial Sequence
  Description of Artificial Sequence Synthetic polynucleotide
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgactgaatggcgaatggcgctttgcctggtttccggcaccagaagcggtgccggaaagctggctggagtgcgatcttcctgacgctcgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacaagacatccagatgacccagtctccatcctccctgtctgcttctgttggggatagagtcaccatcacctgcagggccagtcagagtatcagcagctatctaaattggtaccaacagaaacctggcaaggctcccaagctcctcatctatgctgcatcctctttgcaatcaggcgtcccaagcaggttcagtggcagtgggtctgggacagacttcactctcaccatcagcagtctgcagcctgaagattttgcaacgtattactgtcaacagtcttatagtacaccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgcggccgcacatcatcatcaccatcacggggccgcagaacaaaaactcatctcagaagaggatctgaatggggccgcagaggctagctctgctagtggcgacttcgactacgagaaaatggctaatgccaacaaaggcgccatgactgagaacgctgacgagaatgctttgcaaagcgatgccaagggtaagttagacagcgtcgcgaccgactatggcgccgccatcgacggctttatcggcgatgtcagtggtttggccaacggcaacggagccaccggagacttcgcaggttcgaattctcagatggcccaggttggagatggggacaacagtccgcttatgaacaactttagacagtaccttccgtctcttccgcagagtgtcgagtgccgtccattcgttttcggtgccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 28 - protein, 417 aa, Artificial Sequence
  Description of Artificial Sequence Synthetic polypeptide
MKKLLFAIPLVVPFYSHSAQDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAAAHHHHHHGAAEQKLISEEDLNGAAEASSASGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 29 - DNA, 3457 bp, Artificial Sequence
  Description of Artificial Sequence Synthetic polynucleotide
gacgaaagggcctgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaattgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccctgcaggtctcgggctattcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacatgaagaagctcctctttgctatcccgctcgtcgttccttttgtggcccagccggccatggccgaagttcaattgttagagtctggtggcggtcttgttcagcctggtggttctttacgtctttcttgcgctgcttccggattcactttctctagttacgctatgtcctgggttcgccaagctcctggtaaaggtttggagtgggtttctgctatctctggttctggtggcagtacttactatgctgactccgttaaaggtcgcttcactatctctagagacaactctaagaatactctctacttgcagatgaacagcttaagggctgaggacactgcagtctactattgtgcgagagcctctgcctctaatggtagtgcttacgctgctatagctcctggacttgactactggggccagggaaccctggtcaccgtctcaagcgcctccaccaagggtccgtcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtctagcggactctactccctcagcagcgtagtgaccgtgccctctagcagcttaggcacccagacctacatctgcaacgtgaatcacaagcccagcaacaccaaggtggacaagaaagttgagcccaaatcttgtgcggccgctggtaagcctatccctaaccctctcctcggtctcgattctacgtgataacttcaccggtcaacgcgtgatgagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtctcagtacaatctgctctgatgccgcatagttaagccagccccgacacccgccaacacccgctgacgcgccctgacaggcttgtctgctcccggcatccgcttacagacaagctgtgaccgtctccgggagctgcatgtgtcagaggttttcaccgtcatcaccgaaacgcgcga

SEQ ID NO: 30 - protein, 268 aa, Artificial Sequence
  Description of Artificial Sequence Synthetic polypeptide
MKKLLFAIPLVVPFVAQPAMAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARASASNGSAYAAIAPGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAGKPIPNPLLGLDST

SEQ ID NO: 31 - protein, 286 aa, Artificial Sequence
  Description of Artificial Sequence Synthetic polypeptide
MSIQHFRVALIPFFAAFCLPVFAHPETLVKVKDAEDQLGALVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 32 - DNA, 9030 bp, Artificial Sequence
  Description of Artificial Sequence Synthetic polynucleotide
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgactgaatggcgaatggcgctttgcctggtttccggcaccagaagcggtgccggaaagctggctggagtgcgatcttcctgacgctcgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacagtgatagactagttagacgcgtgcttaaaggcctccaatcctcttggcgcgccaattctatttcaaggagacagtcataatgaaatacctattgcctacggcagccgctggattgttattactcgcggcccagccggccctctgataagatatcacttgtttaaactctgcttggccctcttggccttctagtagacttgcggccgcacatcatcatcaccatcacggggccgcagaacaaaaactcatctcagaagaggatctgaatggggccgcagaggctagctctgctagtggcgacttcgactacgagaaaatggctaatgccaacaaaggcgccatgactgagaacgctgacgagaatgctttgcaaagcgatgccaagggtaagttagacagcgtcgcgaccgactatggcgccgccatcgacggctttatcggcgatgtcagtggtttggccaacggcaacggagccaccggagacttcgcaggttcgaattctcagatggcccaggttggagatggggacaacagtccgcttatgaacaactttagacagtaccttccgtctcttccgcagagtgtcgagtgccgtccattcgttttcggtgccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 33 - DNA, 5957 bp, Artificial Sequence
  Description of Artificial Sequence Synthetic polynucleotide
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacaggtccaactgcaggagctcgagatcaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcaccggtgacaaagagcttcaacaggggagagtgttaataaggcgcgcctaaccatctatttcaaggaacagtcttaatgaaaaagcttttattcatgatcccgttagttgtaccgttcgtggcccagccggcctctgctgaagttcaattgttagagtctggtggcggtcttgttcagcctggtggttctttacgtctttcttgcgctgcttccggagcttcagatctgtttgcctttttgtggggtggtgcagatcgcgttacggagatcgaccgactgcttgagcaaaagccacgcttaactgctgatcaggcatgggatgttattcgccaaaccagtcgtcaggatcttaacctgaggctttttttacctactctgcaagcagcgacatctggtttgacacagagcgatccgcgtcgtcagttggtagaaacattaacacgttgggatggcatcaatttgcttaatgatgatggtaaaacctggcagcagccaggctctgccatcctgaacgtttggctgaccagtatgttgaagcgtaccgtagtggctgccgtacctatgccatttgataagtggtacagcgccagtggctacgaaacaacccaggacggcccaactggttcgctgaatataagtgttggagcaaaaattttgtatgaggcggtgcagggagacaaatcaccaatcccacaggcggttgatctgtttgctgggaaaccacagcaggaggttgtgttggctgcgctggaagatacctgggagactctttccaaacgctatggcaataatgtgagtaactggaaaacaccggcaatggccttaacgttccgggcaaataatttctttggtgtaccgcaggccgcagcggaagaaacgcgtcatcaggcggagtatcaaaaccgtggaacagaaaacgatatgattgttttctcaccaacgacaagcgatcgtcctgtgcttgcctgggatgtggtcgcacccggtcagagtgggtttattgctcccgatggaacagttgataagcactatgaagatcagctgaaaatgtacgaaaattttggccgtaagtcgctctggttaacgaagcaggatgtggaggcgcataaggagttctagagacaactctaagaatactctctacttgcagatgaacagcttaagtctgagcattcggtccgggcaacattctccaaactgaccagacgacacaaacggcttacgctaaatcccgcgcatgggatggtaaagaggtggcgtctttgctggcctggactcatcagatgaaggccaaaaattggcaggagtggacacagcaggcagcgaaacaagcactgaccatcaactggtactatgctgatgtaaacggcaatattggttatgttcatactggtgcttatccagatcgtcaatcaggccatgatccgcgattacccgttcctggtacgggaaaatgggactggaaagggctattgccttttgaaatgaaccctaaggtgtataacccccagcagctagccatattctctcggtcaccgtctcaagcgcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgcagaggctagttctgctagtaacgcgtcttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatgaaacgcgtgatgagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 34 - protein, 6 aa, Artificial Sequence
  Description of Artificial Sequence Synthetic 6xHis tag
HHHHHH

(19) United States
(12) Patent Application Publication
(10) Pub. No.: US 2009/0215119 A1
(43) Pub. Date: Aug. 27, 2009

(54) METHODS FOR PRODUCING SPECIFIC BINDING PAIRS

(75) Inventor: Robert C. Ladner, Ijamsville, MD (US)

Correspondence Address: LANDO & ANASTASI, LLP, ONE MAIN STREET, SUITE 1100, CAMBRIDGE, MA, 02142, US

(73) Assignee: DYAX CORP., Cambridge, MA (US)

(21) Appl. No.: 12/371,000

(22) Filed: Feb. 13, 2009

Related U.S. Application Data
(60) Provisional application No. 61/028,265, filed on Feb. 13, 2008.
(60) Provisional application No. 61/043,938, filed on Apr. 10, 2008.

(51) Int. Cl. C12P 21/00

(52) U.S. Cl. 435/69.6

(57) ABSTRACT

Provided are improved methods for providing specific binding pairs (SBPs). The methods enable production of libraries of SBP members using both a large population of one member of the SBPs and a smaller, preselected population of the other member of the SBPs having affinity for a preselected target.

### CROSS REFERENCE TO RELATED APPLICATIONS

This application claims priority to U.S. application Ser. No. 61/028,265, filed on Feb. 13, 2008 and U.S. application Ser. No. 61/043,938, filed on Apr. 10, 2008. The disclosures of the prior applications are considered part of (and are incorporated by reference in) the disclosure of this application.

### BACKGROUND

Phage display has been known and widely applied in the biological sciences and biotechnology (see, e.g., U.S. Pat. Nos. 5,223,409; 5,403,484; and the references cited therein). The methodology utilizes fusions of nucleic acid sequences encoding foreign polypeptides of interest to sequences encoding phage coat proteins to display the foreign polypeptides on the surface of particles prepared from phage or phagemid. Applications of the technology include the use of affinity interactions to select particular clones from a library of polypeptides, the members of which are displayed on the surfaces of individual phage particles. Display of the polypeptides is due to expression of sequences encoding them from phage vectors into which the sequences have been inserted. Thus, a library of polypeptide encoding sequences is transferred to individual display phage vectors to form a phage library that can be used to select polypeptides of interest.

### SUMMARY

Current methods used for construction of libraries of Fabs and scFvs in phage or phagemid are laborious and inefficient, in part because the combination of Mₕ heavy chains (HCs) with N₁, light chains (LCs) requires Mₕ×N₁ DNA molecules to be constructed and transformed into E. coli. The present method allows the Mₕ HCs to be combined with N₁ LCs through the construction, e.g., of Mₕ (plasmid)+N₁ (phage) novel DNA molecules. The combinatorial mixing is achieved by phage infection which is much more efficient than recombinant ligation of DNA phage or phagemid molecules. The library of N₁ LCs can be reused many times. Hence, to test 10 HC with a population of, for example, 10⁷ LCs requires ten ligations and transformations instead of 10⁸ ligations and transformations. To our knowledge, no one has reported a similar working system nor has anyone discussed the dilution effects that reduce the efficiency of the method if a cellular library is too large.

In the present method, a population of 10⁴ or greater is very likely not to work efficiently because the chance of a selected phage comprising a phage-encoded LC and a cell-derived HC finding a cell that produces the HC that it carried during the selection is lower the larger the HC population used, i.e., because cells are “diluted” in the larger population. Thus, although using a larger number of HCs in the cellular library appears to afford a larger number of possible combinations, the probability of recovering actual binding pairs is lowered due to “dilution”. Because selection by binding can enrich specific binding molecules by between 100 and 1,000-fold per round, we estimate that a cellular library of 100 will function well. Libraries of 20, 10, 6, or less will work better. The method is applicable to a single HC, allowing that HC to be tested with a large number of LCs.

Provided are methods wherein a relatively small number (1 to 1000 (e.g., 1 to 500, 1 to 250, 1 to 100, 1 to 50, 1 to 25, 1 to 15, or e.g., 1, 5, 6, 10, 15, 20, 25, 30, 35, 40, 45, 50, 60, 70, 80, 90, 100, 125, 150, 200, 250, 300, 400, 500, or 750), as opposed to 10⁵ or more) of HCs or LCs with affinity for a preselected target or a particular sequence are combined with a larger, genetically diverse population of LCs or HCs (as appropriate), to produce a library of specific binding pairs, e.g., immunoglobulin fragments such as Fabs.

In some embodiments, 1 to 20 of HCs or LCs with affinity for a preselected target or a particular sequence are combined with a larger, genetically diverse population of LCs or HCs (as appropriate), to produce the library. Examples of other types of specific binding pairs for which the present methods could be used include full length antibodies and antigen-binding fragments thereof (e.g., HC and LC variable domains, Fabs, and so forth), T cell receptor molecules (e.g., the extracellular domains of T cell receptor (TCR) molecules (involving α and β chains, or γ and δ chains)), MHC class I molecules (e.g., involving α1, α2, and α3 domains, non-covalently associated to β2 microglobulin), and MHC class II molecules (involving α and β chains).

In one aspect, in a method termed the Rapid Optimization of LIght Chains or “ROLIC”, a large population of LCs is placed in a phage vector that causes them to be displayed on phage. A small population of HCs (e.g., in a vector, e.g., a plasmid) having specificity for a preselected target are cloned into E. coli so that the HCs are expressed and secreted into the periplasm. The E. coli are then infected with the phage vectors encoding the large population of LCs to produce the HC/LC protein pairings on the phage. The phage particles carry only a LC gene. When a phage particle is selected for binding, the phage must be put back into the cell population from which it came (e.g., the HC-containing E. coli population). The chance that a phage will get into a cell that has the correct HC is inversely proportional to the number of HCs in the population. To improve the efficiency, a population of, for example, 150 HC may be broken up into, for example, 15 populations of 10 subpopulations. Each subpopulation is infected with the whole LC repertoire, the phage are kept segregated, selected in parallel, and each set of phage are returned to the subpopulation from which it came. Thus, the chance of a phage getting into the right cell is increased from 1/150 to 1/10. A LC and HC of interest (e.g., that form a binding pair that binds to a predetermined target) can be isolated from the cell containing them (e.g., by PCR amplification and isolation of the nucleic acids encoding the LC and/or HC of interest), and optionally, rejoined into a standard Fab display format or into a vector for secretion of a soluble Fab (sFab). Either or both of the LC- and HC-containing vectors can contain a selectable marker, e.g., a gene for drug resistance, e.g., kanamycin or ampicillin resistance. Preferably, the plasmid for HC and the phage for LC have different selectable marker genes.

When one or more rounds of selection have been done, one can establish the correct pairing by methods other than PCR. For example, one can cut out the parental LCs from the vectors holding the parental LC-HC pairs and replace them with the newly isolated LCs. One additional round of selection will isolate the LC-HC pairs that bind the target. For example, if there were 8 HCs and one isolated 300 LCs, one would need to do 8 ligations to build the cellular library, and approximately 10⁴ ligations to adequately sample the 8×300 HC-LC combinations.

In another aspect, in a method termed the Economical Selection of Heavy Chains or “ESCH”, a small population of LCs may be placed in a vector (e.g., plasmid) that causes them to be secreted after introduction into E. coli. A new library of HCs in phage is constructed, e.g., the HCs are placed into a phage vector, e.g., that causes the HCs to be displayed on phage. The LCs and HCs can then be combined by the much more efficient method of infection. Once a small set of effective HC are selected, these can be used as is, fed into ROLIC to obtain an optimal HC/LC pairing, or cloned into a Fab library of LCs for classical selection. Either or both of the LC- and HC-containing vectors can contain a selectable marker, e.g., a gene for drug resistance, e.g., kanamycin or ampicillin resistance. Preferably, the plasmid and the phage have different selectable marker genes.

In some aspects, the methods described herein (e.g., ROLIC or ESCH) can be used for affinity maturation of specific binding pairs, such as antibodies. For example, one or several HC or LC from a known antibody that binds to a predetermined target is used in a technique described herein and combined with a library of LC or HC, respectively. The resulting binding pairs are tested for binding to the predetermined target and one or more properties (e.g., binding affinity, amino acid or nucleic acid sequence, the presence of germline sequence, e.g., in a framework region of a variable domain of an antibody or antibody antigen binding fragment, and so forth) can be compared to those of the known antibody. Specific binding pairs with favorable properties (e.g., higher binding affinity to the predetermined target than the known antibody under the same assay conditions) can be evaluated further. See also, Example 4.

These methods establish actual pairings of HC and LC as if a library 10⁵ times larger than the FAB310 or FAB410 libraries (Hoet et al., Nat Biotechnol. 2005 23:344-348) (with on the order of 10¹⁰ members) had been constructed.

In some aspects, the disclosure provides a method of producing specific binding pair (SBP) members with affinity for a predetermined target, wherein the SBP comprises a first polypeptide chain and a second polypeptide chain, which method includes: (i) providing host cells (e.g., E. coli) that comprise, or introducing into host cells, first vectors comprising nucleic acid encoding a first polypeptide chain which has been selected to have affinity for the predetermined target, or a genetically diverse population of said first polypeptide chain all of which have been selected to have affinity for the predetermined target, wherein the first polypeptide chain(s) are secreted from the host cells; and (ii) introducing into the host cells second vectors comprising nucleic acid encoding a genetically diverse population of said second polypeptide chain, wherein the second polypeptide chain is fused to a component of a secreted replicable genetic display package (RGDP) for display of said second polypeptide chains at the surface of RGDPs (e.g., said second vectors being packaged in infectious RGDPs and their introducing into host cells being by infection into host cells harboring said first vectors); (iii) expressing said first and second polypeptide chains within the host cells to form a library of said SBP members displayed by RGDPs, expressing the first and second polypeptide chains within the host cells to form a library of SBP members displayed at the surface of the RGDPs, wherein the first and second polypeptide chains are associated at the surface of the RGDPs; and (iv) selecting members of said population for binding to the predetermined target. Optionally, the method can include infecting a fresh sample of host cells containing the first vectors with the selected RGDPs.

In some embodiments, the first polypeptide chains include antibody heavy chains (HC) or antigen binding fragments thereof.

In some embodiments, the second polypeptide chains include antibody light chains (LC) or antigen binding fragments thereof.

In some embodiments, the first polypeptide chains include antibody light chains (LC) or antigen binding fragments thereof.

In some embodiments, the second polypeptide chains include antibody heavy chains (HC) or antigen binding fragments thereof.

In some embodiments, the first vectors are plasmids.

In some embodiments, the first vectors are phage vectors.

In some embodiments, the second vectors are phage vectors.

In some embodiments, the first vectors encode a genetically diverse population of 1 to 1000 (e.g., 1 to 1000 (e.g., 1 to 500, 1 to 250, 1 to 100, 1 to 50, 1 to 25, 1 to 15, or e.g., 1, 5, 6, 10, 15, 20, 25, 30, 35, 40, 45, 50, 60, 70, 80, 90, 100, 125, 150, 200, 250, 300, 400, 500, or 750) different first polypeptide chains. In some embodiments, the first vectors encode one first polypeptide chain. In some embodiments, the first vectors encode 2 to 1000 (e.g., 2 to 500, 2 to 250, 2 to 100, 2 to 50, 2 to 25, 2 to 15, or e.g., 2, 5, 6, 10, 15, 20, 25, 30, 35, 40, 45, 50, 60, 70, 80, 90, 100, 125, 150, 200, 250, 300, 400, 500, or 750) different first polypeptide chains.

In some embodiments, the first population of vectors encodes 1000 or fewer first polypeptide chains. In some embodiments, the first population of vectors encodes 100 or fewer first polypeptide chains. In some embodiments, the first population of vectors encodes 20 or fewer first polypeptide chains. In some embodiments, the first population of vectors encodes 10 or fewer first polypeptide chains. In some embodiments, the first population of vectors encodes 1 first polypeptide chain.

In some embodiments, the second vectors encode a genetically diverse population of 105 or more different second polypeptide chains.

In some embodiments, the selecting comprises an ELISA (Enzyme-Linked ImmunoSorbent Assay).

In some embodiments, the method futher includes isolating specific binding pair members that bind to the predetermined target.

In some embodiments, the first population is divided into two or more subpopulations and phage produced from one subpopulation are selected and propagated separately from phage produced in other populations.

In some aspects, the disclosure provides a method of producing specific binding pair (SBP) members with affinity for a predetermined target, wherein the SBP comprises a first polypeptide chain and a second polypeptide chain, which method comprises: (i) providing host cells that comprise a first population of vectors comprising a population of genetic material encoding one or more of the first polypeptide chains which have been selected to have one or more desirable properties, wherein the first polypeptide chains are secreted from the host cells; (ii) infecting the cells with a second population of vectors that comprises a diverse population of genetic material that encodes the second polypeptide chains, wherein the second polypeptide chain is fused to a component of a secreted replicable genetic display package (RGDP) for display of the second polypeptide chains at the surface of RGDPs; (iii) expressing the first and second polypeptide chains within the host cells to form a library of SBP members displayed at the surface of the RGDPs, wherein the first and second polypeptide chains are associated at the surface of the RGDPs; and (iv) selecting SBP members for binding to the predetermined target.

In some embodiments, the first polypeptide chains include antibody heavy chains (HC) or antigen binding fragments thereof.

In some embodiments, the second polypeptide chains include antibody light chains (LC) or antigen binding fragments thereof.

In some embodiments, the first polypeptide chains include antibody light chains (LC) or antigen binding fragments thereof.

In some embodiments, the second polypeptide chains include antibody heavy chains (HC) or antigen binding fragments thereof.

In some embodiments, the first vectors are plasmids.

In some embodiments, the first vectors are phage vectors.

In some embodiments, the second vectors are phage vectors.

In some embodiments, the first population of vectors encodes 1 to 1000 (e.g., 1 to 1000 (e.g., 1 to 500, 1 to 250, 1 to 100, 1 to 50, 1 to 25, 1 to 15, or e.g., 1, 5, 6, 10, 15, 20, 25, 30, 35, 40, 45, 50, 60, 70, 80, 90, 100, 125, 150, 200, 250, 300, 400, 500, or 750) different first polypeptide chains. In some embodiments, the first vectors encode one first polypeptide chain. In some embodiments, the first vectors encode 2 to 1000 (e.g., 2 to 500, 2 to 250, 2 to 100, 2 to 50, 2 to 25, 2 to 15, or e.g., 2, 5, 6, 10, 15, 20, 25, 30, 35, 40, 45, 50, 60, 70, 80, 90, 100, 125, 150, 200, 250, 300, 400, 500, or 750) different first polypeptide chains.

In some embodiments, the second vectors encode a genetically diverse population of 10⁵ or more different second polypeptide chains.

In some embodiments, the selecting comprises an ELISA (Enzyme-Linked ImmunoSorbent Assay).

In some embodiments, the method further comprises isolating specific binding pair members that bind to the predetermined target.

In some embodiments, the method further comprises infecting a fresh sample of host cells of step (i) with the selected RGDPs from step (iv).

In some embodiments, the first population is divided into two or more subpopulations and phage produced from one subpopulation are selected and propagated separately from phage produced in other populations.

In some embodiments, the first population of vectors encodes 1000 or fewer first polypeptide chains. In some embodiments, the first population of vectors encodes 100 or fewer first polypeptide chains. In some embodiments, the first population of vectors encodes 20 or fewer first polypeptide chains. In some embodiments, the first population of vectors encodes 10 or fewer first polypeptide chains. In some embodiments, the first population of vectors encodes 1 first polypeptide chain.

In some aspects, the disclosure provides a method of producing specific binding pair (SBP) members with improved affinity for a predetermined target, wherein the SBP comprises a first polypeptide chain and a second polypeptide chain, which method comprises: (i) providing host cells that comprise, or introducing into host cells, a first population of vectors comprising nucleic acid encoding one or more of the first polypeptide chains which have been selected to have affinity for the predetermined target fused to a component of a secreted replicable genetic display package (RGDP) for display of the polypeptide chains at the surface of RGDPs; and (ii) introducing into the host cells a second population of vectors comprising nucleic acid encoding a genetically diverse population of the second polypeptide chain; said first vectors being packaged in infectious RGDPs and their introduction into host cells being by infection into host cells harboring said second vectors; or said second vectors being packaged in infectious RGDPs and their introducing into host cells being by infection into host cells comprising said first vectors; expressing said first and second polypeptide chains within the host cells to form a library of said SBP members displayed by RGDPs, at least one of said populations being expressed from nucleic acid that is capable of being packaged using said RGDP component, whereby the genetic materials of each said RGDP encodes a polypeptide chain of the SBP member displayed at its surface; and selecting members of said population for high-affinity binding to the predetermined target.

In some embodiments, the first population of vectors encodes 1000 or fewer first polypeptide chains. In some embodiments, the first population of vectors encodes 100 or fewer first polypeptide chains. In some embodiments, the first population of vectors encodes 20 or fewer first polypeptide chains. In some embodiments, the first population of vectors encodes 10 or fewer first polypeptide chains. In some embodiments, the first population of vectors encodes 1 first polypeptide chain.

In some embodiments, the first population is divided into two or more subpopulations and phage produced from one subpopulation are selected and propagated separately from phage produced in other populations.

In some aspects, the disclosure provides a method of producing specific binding pair (SBP) members having affinity for a predetermined target, wherein the SBP comprises a first polypeptide chain and a second polypeptide chain, which method comprises: introducing into host cells: (i) first vectors comprising nucleic acid encoding a genetically diverse population of said first polypeptide chain fused to a component of a secreted replicable genetic display package (RGDP) for display of said polypeptide chains at the surface of RGDPs wherein each member of the diverse population is known to have a germline sequence in the framework regions of the variable domain; and (ii) second vectors comprising nucleic acid encoding a genetically diverse population of said second polypeptide chain wherein each member of this population comprises a CDR3 and has synthetic diversity in its CDR3; said first vectors being packaged in infectious RGDPs and their introduction into host cells being by infection into host cells harboring said second vectors; or said second vectors being packaged in infectious RGDPs and their introducing into host cells being by infection into host cells harboring said first vectors; and expressing said first and second polypeptide chains within the host cells to form a library of said SBP members displayed by RGDPs, at least one of said populations being expressed from nucleic acid that is capable of being packaged using said RGDP component, whereby the genetic materials of each said RGDP encodes a polypeptide chain of the SBP member displayed at its surface.

Compositions and kits for the practice of these methods are also described herein. These embodiments of the present invention, other embodiments, and their features and characteristics will be apparent from the description, drawings, and claims that follow.

### BRIEF DESCRIPTION OF THE DRAWINGS

FIG. 1 depicts an embodiment of the ROLIC method described in EXAMPLE 1.

FIG. 2 depicts an exemplary ROLIC LC selection scheme (right) compared to a conventional phage selection scheme (left), illustrating the better efficiency and pairing rate of ROLIC, as well as removal of the requirement of a library to achieve a high potential number of pairings.

FIG. 3 depicts how incorporating ROLIC into a selection/screening method reduces the number of steps in the method.

FIG. 4 depicts the results of a cell strain evaluation for XL1 Blue MRF and other cell lines, as described in EXAMPLE 1.

FIG. 5 depicts an exemplary HC vector to be used in a ROLIC method.

FIG. 6 depicts the results of an ELISA analyzing whether 20 light chains in DY3F85 LC can pair with the 20 heavy chains in pHCSK22 to create a functional Fab on phage, as described in EXAMPLE 1.

FIG. 7 depicts the results of an ELISA analyzing whether 20 light chains in DY3F85 LC can pair with the 20 heavy chains in pHCSK22 to create a functional Fab on phage, as described in EXAMPLE 1.

FIG. 8 depicts the results of an ELISA comparison of phage titer and display.

FIG. 9 depicts the results of an ELISA analyzing whether ROLIC selection works with full light chain diversity and 20 anti-Tie1 heavy chains (4e7 LC×20 HC).

FIG. 10 depicts the results of an ELISA analyzing whether ROLIC selection works with full light chain diversity and 20 anti-Tie1 heavy chains (4e7 LC×20 HC).

FIG. 11 depicts the results of an ELISA analyzing whether ROLIC selection works with full light chain diversity and 20 anti-Tie1 heavy chains (4e7 LC×20 HC).

FIG. 12 depicts the results of an ELISA analyzing whether ROLIC selection works with full light chain diversity and 20 anti-Tie1 heavy chains (4e7 LC×20 HC).

FIG. 13 depicts the results of an ELISA analyzing whether ROLIC selection works with full light chain diversity and 20 anti-Tie1 heavy chains (4e7 LC×20 HC).

FIG. 14 summarizes the results of ELISAs analyzing whether ROLIC selection works with full light chain diversity and 20 anti-Tie1 heavy chains (4e7 LC×20 HC).

FIG. 15 is a design overview of a “zipping” method to relink VH and VL-CL after a ROLIC selection, as described in EXAMPLE 2. LC-DY3P85 is identical to DY3F85LC. If the cassette is cloned into pMID21, we obtain display phagemid. If the cassette is cloned into pMID21.03, we obtain a vector for sFab expression.

FIG. 16 depicts a SDS-PAGE illustrating successful use of a “zipping” method as described in EXAMPLE 2.

### DETAILED DESCRIPTION

For convenience, before further description of the present invention, certain terms employed in the specification, examples and appended claims are defined here.

The singular forms “a”, “an”, and “the” include plural references unless the context clearly dictates otherwise.

The term “affinity” or “binding affinity” refers to the apparent association constant or Ka. The Kₐ is the reciprocal of the dissociation constant (K_{d}). A binding protein may, for example, have a binding affinity of at least 10⁵, 10⁶, 10⁷, 10⁸, 10⁹, 10 and 10¹¹ M⁻¹ for a particular target molecule. Higher affinity binding of a binding protein to a first target relative to a second target can be indicated by a higher Kₐ (or a smaller numerical value K_{d}) for binding the first target than the Kₐ (or numerical value K_{d}) for binding the second target. In such cases, the binding protein has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, or 10⁵ fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in TRIS-buffer (50 mM TRIS, 150 mM NaCl, 5 mM CaCl₂ at pH7.5). These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

[Bound]=N.[Free]/((1/Ka)+[Free]).

It is not always necessary to make an exact determination of Kₐ, though, since sometimes it is sufficient to obtain a qualitative or semi-quantitative measurement of affinity, e.g., determined using a method such as ELISA or FACS analysis, is proportional to Kₐ, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J Immunol. 1996; 26(3):629-39.)) as well as complete antibodies. An antibody can have the structural features of IgA, IgG, IgE, IgD, IgM (as well as subtypes thereof). Antibodies may be from any source, but primate (human and non-human primate) and primatized are preferred.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin may be of types, kappa or lambda. In one embodiment, the antibody is glycosylated. An antibody can be functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be converted to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, CL1), or the entire antibody can be human or effectively human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH-terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

A “library” refers to a collection of nucleotide, e.g., DNA, sequences within clones; or a genetically diverse collection of polypeptides, or specific binding pair (SBP) members, or polypeptides or SBP members displayed on RGDPs capable of selection or screening to provide an individual polypeptide or SBP members or a mixed population of polypeptides or SBP members.

The term “package” as used herein refers to a replicable genetic display package in which the particle is displaying a member of a specific binding pair at its surface. The package may be a bacteriophage which displays an antigen binding domain at its surface. This type of package has been called a phage antibody (pAb).

A “pre-determined target” refers to a target molecule whose identity is known prior to using it in any of the disclosed methods.

The term “replicable genetic display package (RGDP)” as used herein refers to a biological particle which has genetic information providing the particle with the ability to replicate. The particle can display on its surface at least part of a polypeptide. The polypeptide can be encoded by genetic information native to the particle and/or artificially placed into the particle or an ancestor of it. The displayed polypeptide may be any member of a specific binding pair e.g., heavy or light chain domains based on an immunoglobulin molecule, an enzyme or a receptor etc. The particle may be, for example, a virus e.g., a bacteriophage such as fd or M13.

The term “secreted” refers to a RGDP or molecule that associates with the member of a SBP displayed on the RGDP, in which the SBP member and/or the molecule, have been folded and the package assembled externally to the cellular cytosol.

The term “specific binding pair (SBP)” as used herein refers to a pair of molecules (each being a member of a specific binding pair) which are naturally derived or synthetically produced. One of the pair of molecules, has an area on its surface, or a cavity which specifically binds to, and is therefore defined as complementary with a particular spatial and polar organization of the other molecule, so that the pair have the property of binding specifically to each other. Examples of types of specific binding pairs are antigen-antibody, biotin-avidin, hormone-hormone receptor, receptor-ligand, enzyme-substrate, IgG-protein A.

The term “vector” refers to a DNA molecule, capable of replication in a host organism, into which a gene is inserted to construct a recombinant DNA molecule. A “phage vector” is a vector derived by modification of a phage genome, containing an origin of replication for a bacteriophage, but not one for a plasmid. A “phagemid vector” is a vector derived by modification of a plasmid genome, containing an origin of replication for a bacteriophage as well as the plasmid origin of replication. Phagemid vectors offer the convenience of cloning into a vector that is much smaller than a display phage; phagemid infected cells must be rescued with helper phage.

In one aspect, provided is a method of producing specific binding pair (SBP) members with affinity for a predetermined target, wherein the SBP comprises a first polypeptide chain and a second polypeptide chain, which method comprises: (i) providing a population of host cells (e.g., E. coli) harboring a first vector containing a population of genes encoding one or more of the first polypeptide chains all of which have been selected to have one or more desirable properties, wherein the first polypeptide chains are secreted from the host cells; (ii) infecting the host cells with a population of second vectors, wherein the population of second vectors encodes a population (e.g., genetically diverse population) of the second polypeptide chains, wherein the second polypeptide chain is fused to a component of a secreted replicable genetic display package (RGDP) for display of the second polypeptide chains at the surface of RGDPs; (iii) expressing the first and second polypeptide chains within the cells to form a library of SBP members displayed by RGDPs, whereby the genetic material of each said RGDP encodes a polypeptide chain of said second population of the SBP member displayed at its surface; (iv) selecting members of said population for binding to the predetermined target; and optionally, (v) infecting a fresh sample of the population of host cells of step (i) with the selected RGDPs.

In one aspect, provided is a method of producing specific binding pair (SBP) members with improved affinity for a predetermined target comprising a first polypeptide chain and a second polypeptide chain that comprises: introducing into host cells; (i) first vectors comprising nucleic acid encoding a genetically diverse population of said first polypeptide chain all of which have been selected to have one or more desirable properties wherein the gene for each said first polypeptide chain is operably linked to a signal sequence so that said polypeptide chain is secreted into the periplasm as a soluble molecule; and (ii) second vectors comprising nucleic acid encoding a genetically diverse population of said second polypeptide chain fused to a component of a secreted replicable genetic display package (RGDP) for display of said polypeptide chains at the surface of RGDPs; said second vectors being packaged in infectious RGDPs and their introduction into host cells being by infection into host cells harboring said first vectors. The desirable properties for which the first population might be selected include: a) having affinity for a predetermined target, b) encoding germline amino-acid sequence in the framework regions, c) having optimal codon usage for E. coli, d) having optimal codon usage for CHO cells, e) being devoid of particular restriction enzyme recognition sites, and f) having synthetic or selected diversity in one or more CDRs (e.g., HC CDR1, HC CDR2, HC CDR3, LC CDR1, LC CDR2, and/or LC CDR3). In some embodiments, the synthetic or selected diversity is in HC CDR3.

The predetermined target may be any target of interest, for example, a target for therapeutic intervention, e.g., Tie-1, MMP-14, MMP-2, MMP-12, MMP-9, FcRN, VEGF, TNF-alpha, plasma kallikrein, etc. Affinity for a particular target may be determined by any method as is known to one of skill in the art.

In certain embodiments, the first polypeptide chain includes a LC or HC, and the second polypeptide chain a LC or HC depending on what the identity of the first polypeptide contains. For example, in embodiments where the first polypeptide chain includes a LC, the second polypeptide includes a HC. In other embodiments, where the first polypeptide chain includes a HC, the second polypeptide chain includes a LC.

The genetically diverse population of the first polypeptide chain, all of which have been selected to have a desirable property, may comprise at least about 5, about 10, about 25, about 50, about 75, about 100, about 200, about 300, about 400, about 500, about 750, to about 1000 members. The genetically diverse population of the second polypeptide chain is generally much larger, on the order of at least about 10⁵, 10⁶, 10⁷ or greater.

In certain embodiments, each or either said polypeptide chain may be expressed from nucleic acid which is capable of being packaged as a RGDP using said component fusion product.

The method may comprise introducing vectors capable of expressing a population of said first polypeptide chains into host organisms under conditions that suppress said expression. Into this population of cells, under conditions that allow expression of both the first and second polypeptide chains, are introduced phage vectors capable of causing expression of said second polypeptide chain as a fusion to a coat protein of the phage vector.

When a phage is used as RGDP it may be selected from the class I phages fd, M13, f1, If1, lke, ZJ/Z, Ff and the class II phages Xf, Pf1 and Pf3. In certain embodiments, the filamentous F-specific bacteriophages may be used to provide a vehicle for the display of binding molecules e.g., antibodies and antibody fragments and derivatives thereof, on their surface and facilitate subsequent selection and manipulation. The single stranded DNA genome (approximately 6.4 Kb) of fd is extruded through the bacterial membrane where it sequesters capsid sub-units, to produce mature virions. These virions are 6 nm in diameter, 1 μm in length and each contain approximately 2,800 molecules of the major coat protein encoded by viral gene VIII and four molecules of the adsorption molecule gene III protein (g3p) the latter is located at one end of the virion. The structure has been reviewed by Webster et al., 1978 in The Single Stranded DNA Phages, 557-569, Cold Spring Harbor Laboratory Press. The gene III product is involved in the binding of the phage to the bacterial F-pilus. It has been recognized that gene III of phage fd is an attractive possibility for the insertion of biologically active foreign sequences. There are however, other candidate sites including for example gene VIII and gene VI. In certain embodiments, the gene III stump is used in the methods herein.

Host cells may be any host cell capable of being infected by phage. In certain embodiments, the host cell is a strain of E. coli, e.g.,TG1, XL1 Blue MRF′, Ecloni or Top10F′.

Following combination RGDPs may be selected or screened to provide an individual SBP member or a mixed population of said SBP members associated in their respective RGDPs with nucleic acid encoding a polypeptide chain thereof. The restricted population of at least one type of polypeptide chain provided in this way may then be used in a further dual combinational method in selection of an individual, or a restricted population of complementary chain.

Nucleic acid taken from a restricted RGDP population encoding said first polypeptide chains may be introduced into a recombinant vector into which nucleic acid from a genetically diverse repertoire of nucleic acid encoding said second polypeptide chains is also introduced, or the nucleic acid taken from a restricted RGDP population encoding said second polypeptide chains may be introduced into a recombinant vector into which nucleic acid from a genetically diverse repertoire of nucleic acid encoding said first polypeptide chains is also introduced.

The recombinant vector may be produced by intracellular recombination between two vectors and this may be promoted by inclusion in the vectors of sequences at which site-specific recombination will occur, such as loxP sequences obtainable from coliphage P1. Site-specific recombination may then be catalyzed by Cre-recombinase, also obtainable from coliphage P1.

The Cre-recombinase used may be expressible under the control of a regulatable promoter.

In another aspect, a method of producing specific binding pair (SBP) members having affinity for a predetermined target comprising a first polypeptide chain and a second polypeptide chain comprises: introducing into host cells; (i) first vectors comprising nucleic acid encoding a genetically diverse population of said first polypeptide chain wherein each member of the diverse population is known to have a germline sequence in the framework regions of the variable domain; and (ii) second vectors comprising nucleic acid encoding a genetically diverse population of said second polypeptide chain wherein each member of this population has synthetic diversity in its CDR3 and said second polypeptide chain is fused to a component of a secreted replicable genetic display package (RGDP) for display of said polypeptide chains at the surface of RGDPs; said second vectors being packaged in infectious RGDPs and their introduction into host cells being by infection into host cells harboring said first vectors.

Human germline sequences are disclosed in Tomlinson, I. A. et al., 1992, J. Mol. Biol. 227:776-798; Cook, G. P. et al., 1995, Immunol. Today Vol.16 (5): 237-242; Chothia, D. et al., 1992, J. Mol. Bio. 227:799-817. The V BASE directory provides a comprehensive directory of human immunoglobulin variable region sequences (compiled by Tomlinson, I. A. et al. MRC Centre for Protein Engineering, Cambridge, UK). Antibodies are “germlined” by reverting one or more non-germline amino acids in framework regions to corresponding germline amino acids of the antibody, so long as binding properties are substantially retained. Similar methods can also be used in the constant region, e.g., in constant immunoglobulin domains.

Antibodies may be modified in order to make the variable regions of the antibody more similar to one or more germline sequences. For example, an antibody can include one, two, three, or more amino acid substitutions, e.g., in a framework, CDR, or constant region, to make it more similar to a reference germline sequence. One exemplary germlining method can include identifying one or more germline sequences that are similar (e.g., most similar in a particular database) to the sequence of the isolated antibody. Mutations (at the amino acid level) are then made in the isolated antibody, either incrementally or in combination with other mutations. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a framework and/or constant region. For example, a germline framework and/or constant region residue can be from a germline sequence that is similar (e.g., most similar) to the non-variable region being modified. After mutagenesis, activity (e.g., binding or other functional activity) of the antibody can be evaluated to determine if the germline residue or residues are tolerated (i.e., do not abrogate activity). Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations more than one or two germline sequences are used, e.g., to form a consensus sequence.

Also provided are kits for use in carrying out a method according to any aspect of the invention. The kits may include the necessary vectors. One such vector will typically have an origin of replication for single stranded bacteriophage and either contain the SBP member nucleic acid or have a restriction site for its insertion in the 5′ end region of the mature coding sequence of a phage capsid protein, and with a secretory leader coding sequence upstream of said site which directs a fusion of the capsid protein exogenous polypeptide to the periplasmic space.

Also provided are RGDPs as defined above and members of specific binding pairs e.g., binding molecules such as antibodies, enzymes, receptors., fragments and derivatives thereof, obtainable by use of any of the above defined methods. The derivatives may comprise members of the specific binding pairs fused to another molecule such as an enzyme or a Fc tail.

The kit may include a phage vector (e.g., DY3F85LC, sequence in Table 2) which may have the above characteristics, or may contain, or have a site for insertion, of SBP member nucleic acid for expression of the encoded polypeptide in free form. The kit may also include a plasmid vector for expression of the soluble chain, e.g., pHCSK22 (sequence in Table 3). The kit may also include a suitable cell line (e.g., TG1).

The kits may include ancillary components required for carrying out the method, the nature of such components depending of course on the particular method employed. Useful ancillary components may comprise helper phage, PCR primers, and buffers and enzymes of various kinds. Buffers and enzymes are typically used to enable preparation of nucleotide sequences encoding Fv, scFv or Fab fragments derived from rearranged or unrearranged immunoglobulin genes according to the strategies described herein.

### EXEMPLIFICATION

The present invention is further illustrated by the following examples which should not be construed as limiting in any way. The contents of all references, pending patent applications and published patents, cited throughout this application are hereby expressly incorporated by reference.

### EXAMPLE 1: Rapid Optimization of LIght Chains (ROLIC)

ROLIC is the Rapid Optimization of LIght Chains. In an exemplary embodiment of this method, the genes encoding a population of SS-VH(i)-CH1 are placed in a vector (such as pHCSK22) under control of a suitable regulatable promoter, such as PlacZ. SS is a signal sequence that will cause secretion of VH(i)-CH1 in E. coli (i is the index of this VH in the population, i could be 1,2, . . . N). VH(i) is a variable domain of a heavy chain of an antibody and CH1 is the first constant domain of an IgG heavy chain (HC). The vector pHCSK22 also contains the origin of replication of pBR322 and a kanamycin resistance gene (kanR). The HC population put into pHCSK22 will have been selected to have affinity for a particular target antigen or for some other desirable property.

A second vector, DY3F85LC, is a phage derived vector from M13mpl8. In addition to all the genes of wild-type M13, DY3F85LC carries an ampicillin resistance gene (bla) and a display cassette for antibody light chains (LC). The LC constant region is fused in-frame to the stump of M13 iii. The SS-VL-CL-IIIstump gene is regulated by PlacZ. A large repertoire of human LCs is cloned into DY3F85LC.

In one example, 20 HCs having affinity for human TIE-1 are cloned into pHCSK22 and used to transform TGI E. coli to make a cell population. These cells are F+and can be infected with M13. When a cell harbors both one member of the pHCSK22 population and one member of the DY3F85LC population, the cell is resistant to both Amp and Kan. When induced with IPTG or when grown in the absence of glucose, HCs are secreted into the periplasm, each cell making one member of the HC population. M13 have a well developed system to avoid multiple infection, so that each cell contains a single member of the LC population. Thus, the phage produced from Amp^{R}, Kan^{R} cells will carry the gene for the LC that is anchored to the IIIₛₜᵤₘₚ. Because DY3F85LC has both w.t. iii and the display vl::cl::iiiₛₜᵤₘₚ, the phage will have mostly full-length III. Many phage will have only w.t. III and no antibody display. Phage that do carry a VL::CL::IIIₛₜᵤₘₚ protein will obtain a VH::CH1 protein from the periplasm of the cell.

If there are, for example, 5×10⁷ LCs and 20 distinct HCs, there could be 10⁹ LC/HC combinations. These phage can be selected for binding to the target, e.g., TIE-1. In the original FAB-310 library, each HC was paired with approximately 25 different LCs. Here we take a small set of HC, all of which have some affinity for TIE-1 and combine them with all the LCs in our collection. While it would be possible to make a library of 10⁹ in our vector pMID22, making a library of this size is highly labor intensive. In ROLIC, we need make only the library of 20 HC in pHCSK22 and transform E. coli cells. The infection of these cells with the DY3F85LC library allows the full combination. The DY3F85LC library need be built but once.

Phage that are selected for binding must be propagated in the same cell line from which they were obtained because they do not carry the HC gene. Cells (carrying the HC population) infected with the selected LC phage are grown in liquid overnight. The amplified phage are precipitated, purified, and exposed to the target in question. Target bound by phage are mixed with the original HC pHCSK22 bacteria which allows for infection and amplification of the phage and potentially new LC HC pairings. This process is repeated 2 or 3 times until eventually the cells containing the phage are plated. Individual colonies are picked and grown. Phage from isolated colonies (e.g., 960) are tested in a phage ELISA. In the colonies that produce phage that bind the target, we have the desired pairing, although the LC and HC genes are on separate DNA molecules. Using PCR, we can rejoin LC and HC into the standard Fab display format as described in Hoet, R.M. et al. Nat Biotechnol 23, 344-348 (2005). Alternatively, we could produce a soluble Fab (sFab) expression cassette and test sFabs.

ROLIC allows us to affinity mature 1 to 100 (or even 1 to 500) antibodies at one time. We are not forced to pick one antibody with the risk that there is not a better LC in the available repertoire. If we originally select antibodies that have affinities in the range 100 pM to 100 nM and one third of these show a ten fold improvement, then we should have antibodies with affinities in the range 20 pM to 100 nM for very little additional effort.

A. Exemplary ROLIC Method

1. Select 1-2 rounds from FAB-310 or FAB-410.

2. Move the HCs in a population of plasmids into a cell library as untethered HCs (HC repertoire of 1-1000; little or no characterization).

3. Infect the cell library with a phage library carrying 5 E⁷ kappas & 5 E⁷ lambdas anchored to IIIₛₜᵤₘₚ and no HC.

4. Select phage, repeat once (use same cellular library).

5. Use phage ELISAs to pick colonies that harbor a working LC/HC pair.

6. Construct sFab cassettes from ELISA-positive colonies in pMID21.03. (pMID21.03 is a vector derived from pMID21 in which the IIIstump is deleted so that sFabs are secreted.)

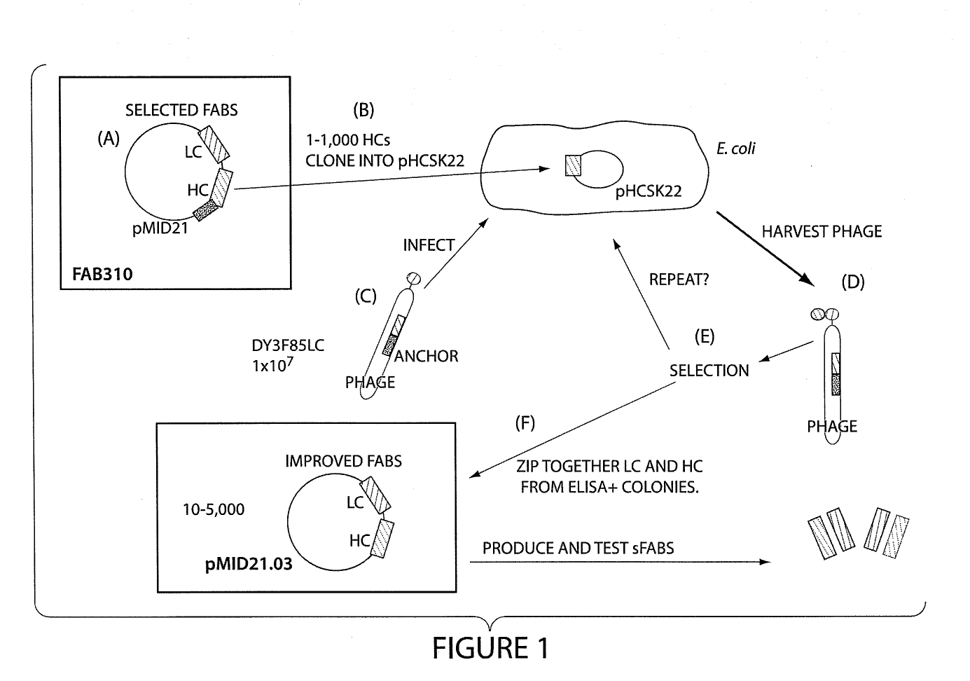

This method establishes actual pairings of HC and LC as if the library were 10⁵ times larger than FAB-310 or FAB-410. It is illustrated in FIG. 1. At step 2 above, one need not characterize the HC to any preset degree. One is free to pick HCs that all exhibit a desirable feature, such as inhibiting an enzyme. The phage library FAB-410 was built in the phage vector DY3F63, shown in Table 4. The phagemid library FAB-310 was built in the phagemid vector pMID21, shown in Table 5.

B. Selecting LCs—Examples

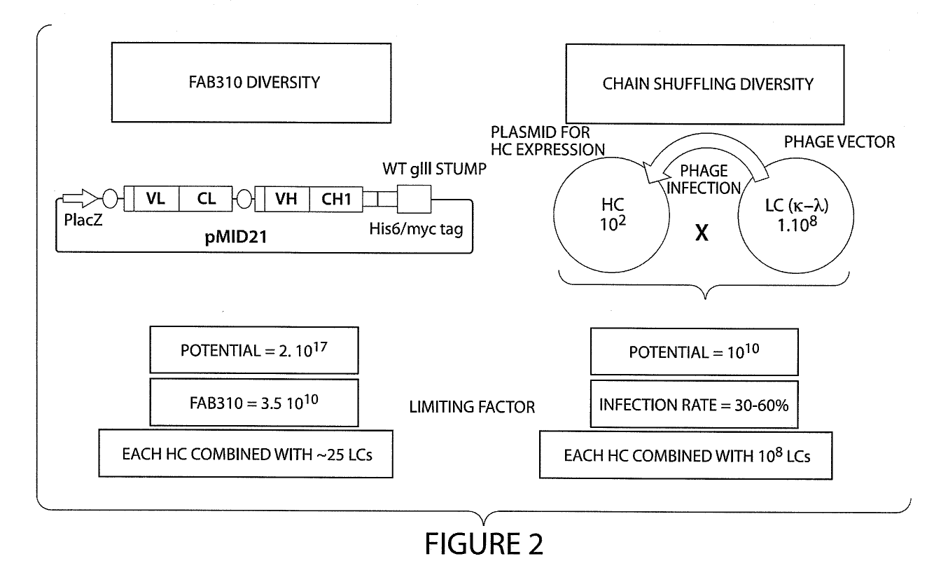

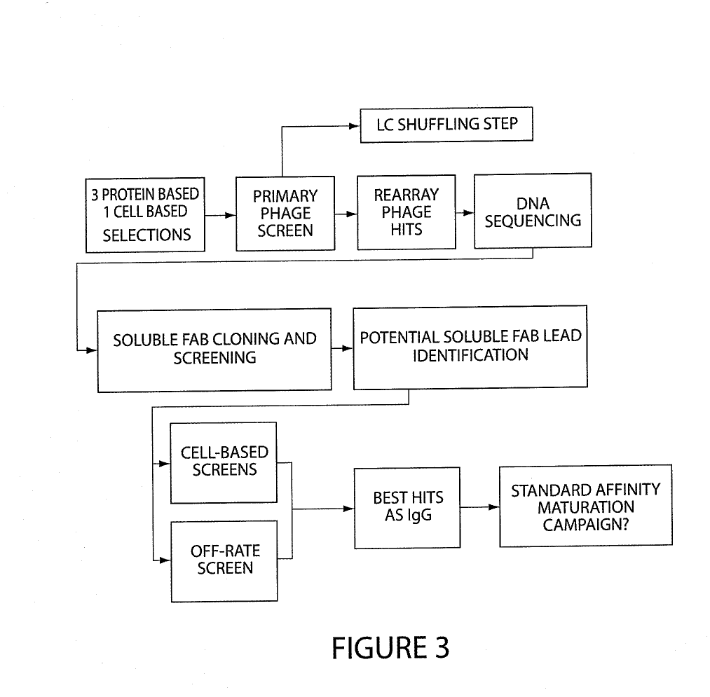

FIG. 2 illustrates one method of selecting LCs using ROLIC. FIG. 3 illustrates a potentially faster method.

C. Kappa and Lambda LC Library Construction

Before building a full library, the following evaluation experiments were completed:

1. K and λ LCs were ligated into a DY3F85LC vector on a small scale

2. 20 ng of the final vector was electroporated into XL1 Blue cells and plated

3. 4 plates were picked for each library

4. We confirmed that LCs are expressed on the phage (k & X LC ELISA)

5. Diversity of each library was evaluated by sequencing 4 plates for each library

6. 3 E. coli strains were evaluated

Two anti -human LC antibodies were tested for each library—rabbit and goat. Kappa and lambda LC from pMID17 were successfully displayed on DY3F85LC phage, allowing construction of a large light chain library. The vector pMID17 is a holding vector for LC-HC Ab (antibody) cassettes and contains a bla gene but lacks a display anchor.

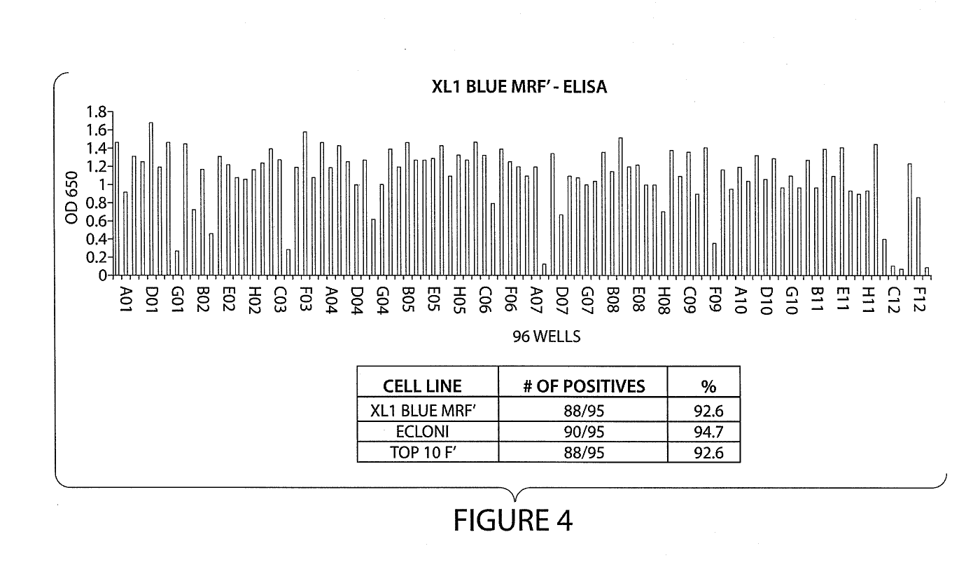

Three E. coli strains were evaluated: XL1 Blue MRF′ (Stratagene), Ecloni (Lucigen) and Top10F′ (Invitrogen). The following parameters were tested: kappa LC expression (ELISA), transformation efficiency (titer) and ability to produce phage (phage purification and titer). FIG. 4 depicts the results of the ELISA evaluation of kappa LC expression in the three strains. The transformation efficiency of each strain was as follows: XL1 Blue MRF′—7.3×10⁶ CFU/μg, Ecloni—4.3×10⁶ CFU/μg and Top10 F′—6.8×10⁶ CFU/μg. The purified phage titer measurements were as follows:

PFU: XL1 Blue MRF′—3.58×10⁹; Ecloni—1.56×10⁹ and Top10 F′—5.07×10⁹

CFU: XL1 Blue MRF′—1.19×10⁹; Ecloni—5.36×10⁸ and Top10 F′—6.30×10⁸

The light chain expression, efficiency of transformation and ability to produce phage was comparable for all the tested E. coli strains.

XL1 Blue MRF′ was chosen to create a large library. The steps/parameters comprising the large library construction were:

1. Test ligations

2. Large scale ligations (×60)

3. Test electroporations (EPs)

4. Large scale EPs (60 EPs per library)

5. Titer (Library size): Kappa—2×10⁷ total CFU and Lambda—1×10⁷ total CFU

6. NUNC plating/scraping

7. PEG precipitation and phage purification

8. Final Titer: Kappa—6×10⁷/μL and Lambda—8×10⁶/μL

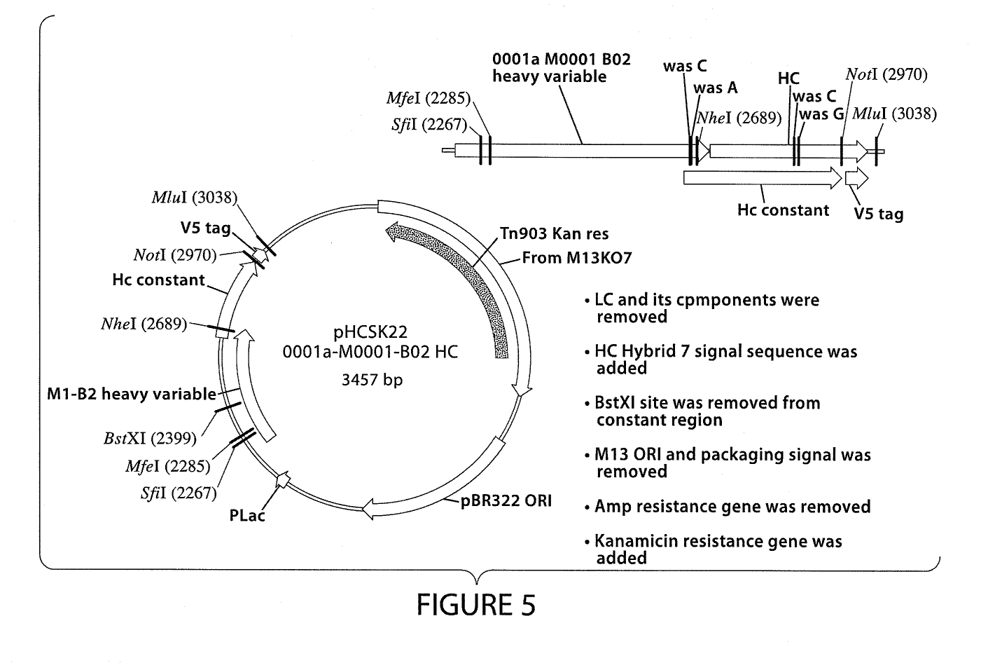

The HC vector used to express and pair HCs with the LC library, and information on its construction, is shown in FIG. 5.

D. Proof-of Conceptfor ROLIC

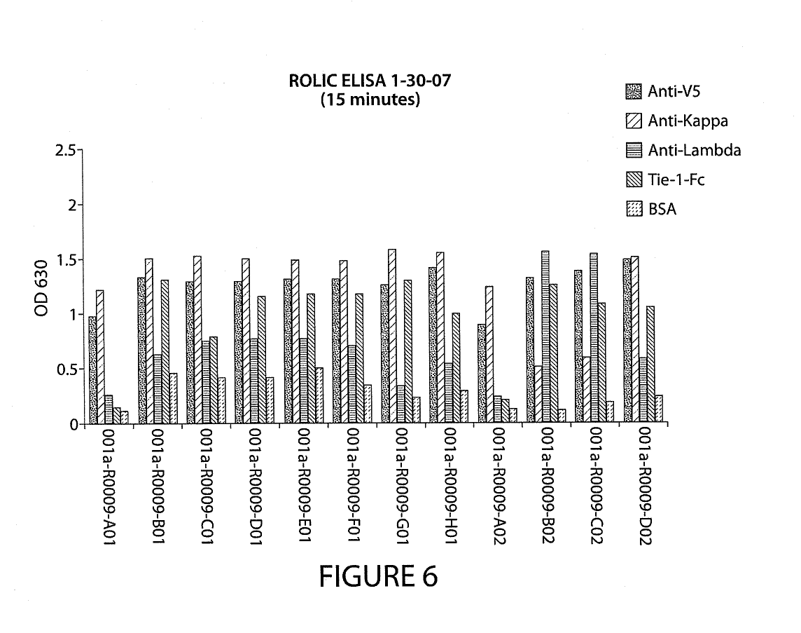

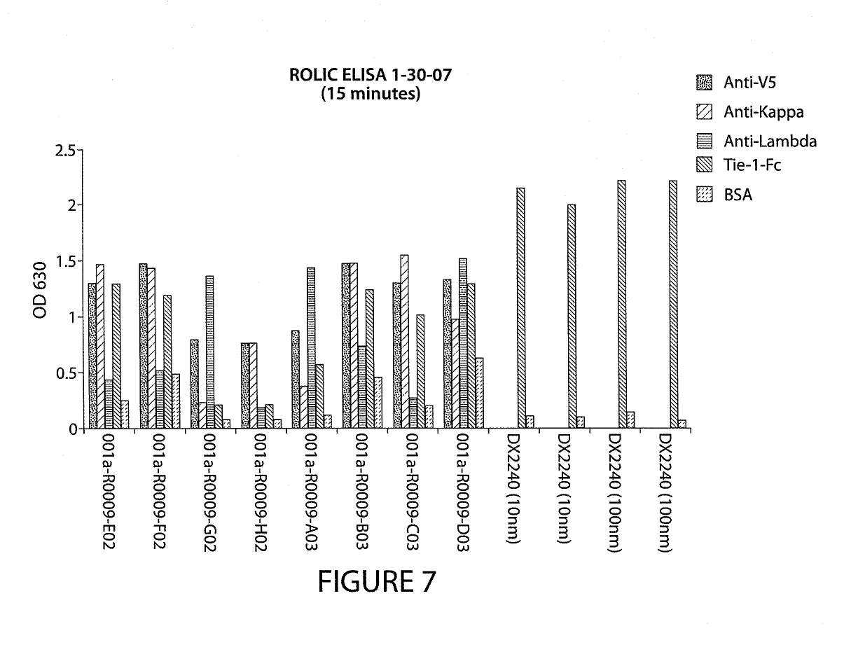

Twenty HCs having specificity for Tie-I were chosen for proof-of-concept experiments. Anti-Tie-1 and anti-heavy chain (V5) and anti-light chain ELISAs were used to evaluate whether the 20 light chains in DY3F85LC could pair with the 20 heavy chains in pHCSK22 to create a functional Fab on phage (1 LC×1 HC). Exemplary results of the ELISAs are shown in FIGS. 6 and 7, indicating that the LCs could pair with HCs to create Fabs (having both LCs and HCs) with anti-Tie-1 activity.

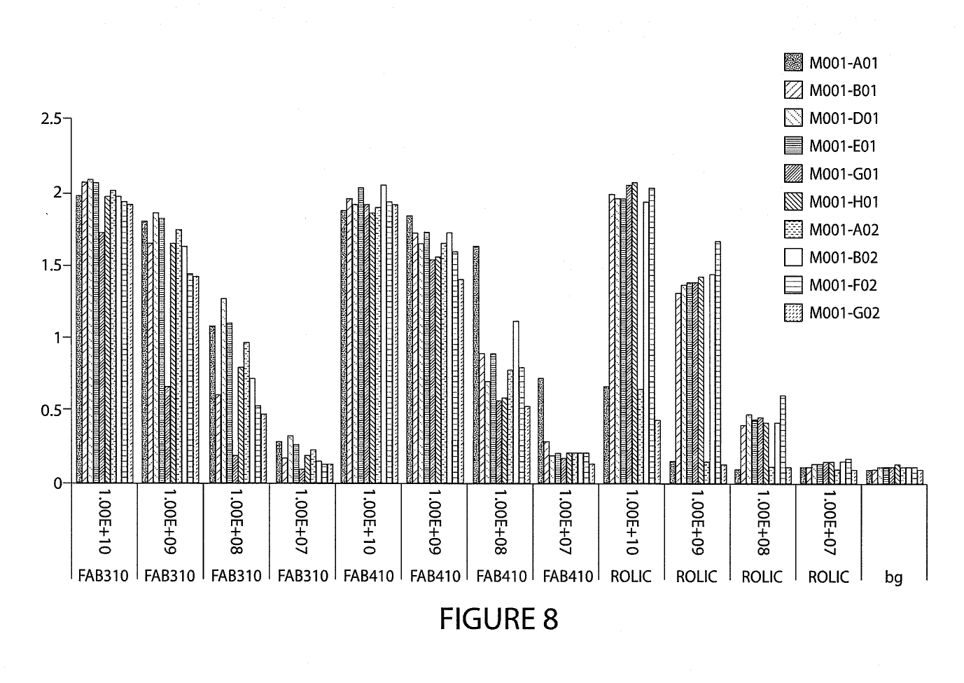

A comparison of the display from this library to that of pMID21 and DY3F63 (Fab310 and Fab410) was performed using anti-Tie1 ELISA titrations and anti-Fab (or HC and LC specific) ELISA titrations. Specifically, the anti-Tie1 ELISAs were performed as follows. Ten individual Tie-1 HC-pHCSK22 clones with their corresponding (original) 10 individual Tie-1 LC-DY3F85LC were rescued and incubated overnight at 30° C. The phage were PEG precipitated and phage titration (CFU) performed. The ELISA was performed as follows: 1) Coat a 96 well plates with anti-Fab antibody (1 μg/mL, 100 ul/well in PBS), overnight (O/N) at 4° C., 2) Block with 4% BSA in PBS, 1 hr room temperature (RT), 3) Wash with PBST (0.1% TWEEN® 20), 4) Add phage to wells, incubate 1 hr at RT, 4) Wash with PBST (0.1% TWEEN® 20), 5) Add anti-M13-HRP, incubate 1 hr at RT, 6) Wash, add substrate and 6) read at 450 nm. The comparison of phage titer and display among the libraries is shown in FIG. 8.

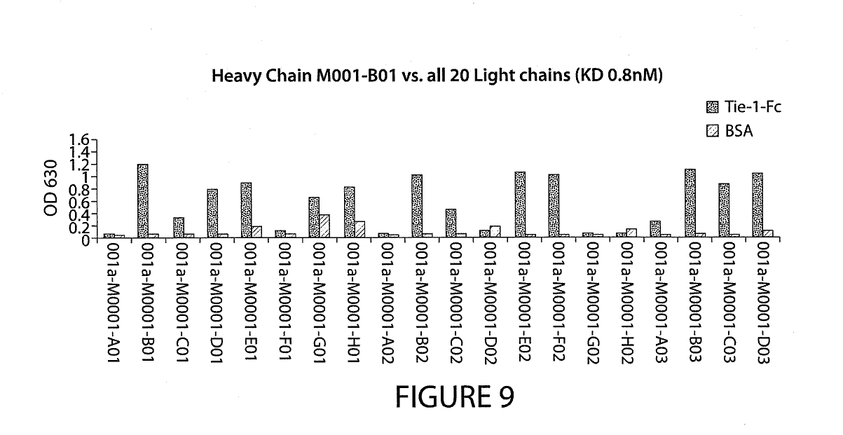

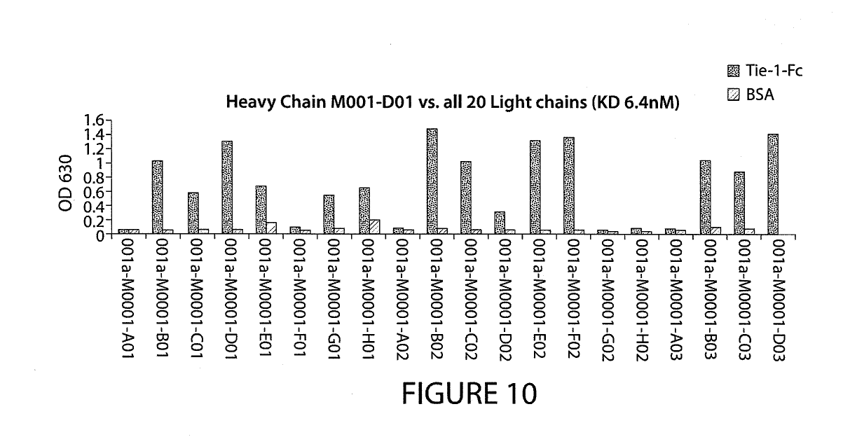

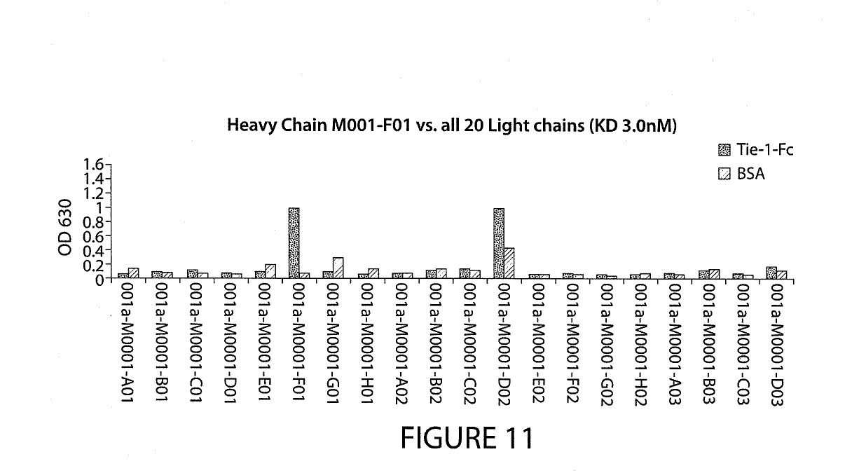

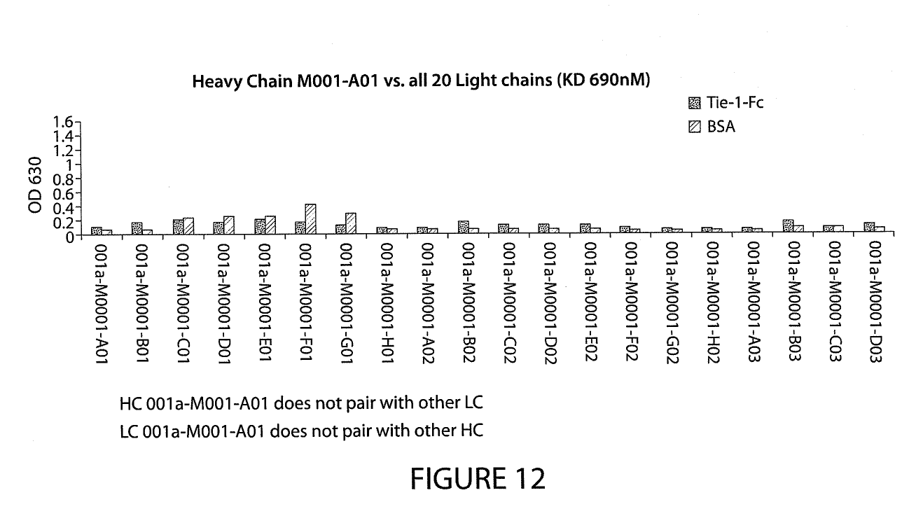

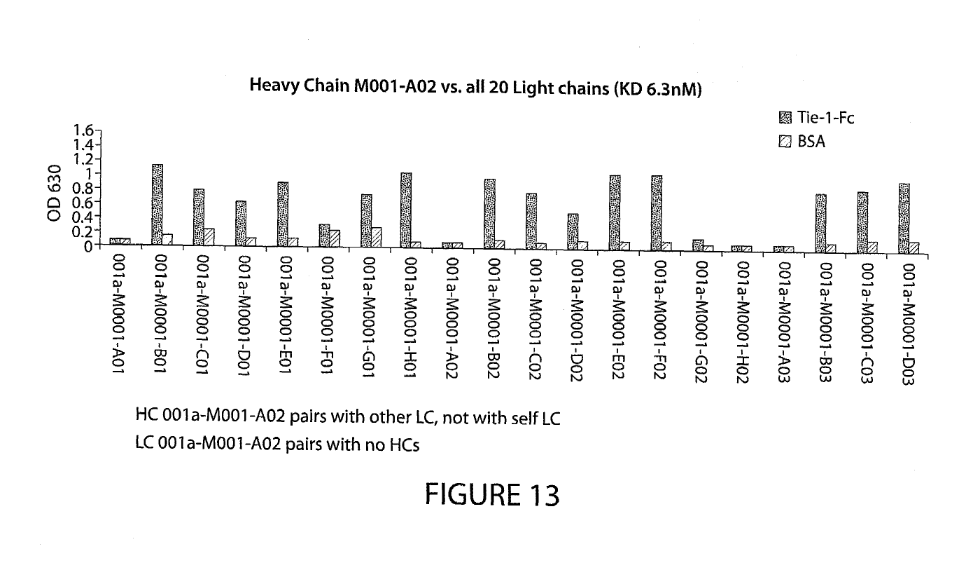

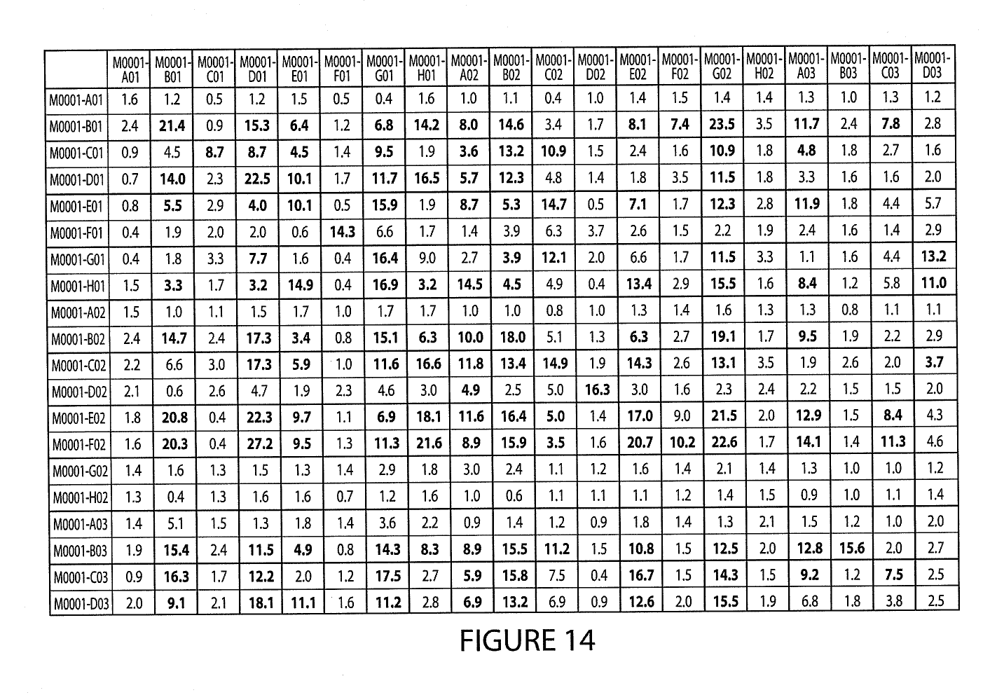

We then evaluated whether a ROLIC selection works with a mixed population of anti-Tie1 light chains and heavy chains ((20 LC×1 HC) or (20 LC×20 HC)). Tie-1 HC-pHCSK22 clones were rescued with Tie1 LC-DY3F85LC, the results of which were analyzed with an anti-Tie-1 ELISA and sequencing. Exemplary results are shown in FIGS. 9 through 13, with a summary table in FIG. 14.

Whether a ROLIC selection works with full light chain diversity and the 20 anti-Tie1 heavy chains (4e7 LC×20 HC) was determined by rescuing Tie1 Hc-pHCSK22 clones with K-DY3F85LC and L-DY3F85LC, the results of which were analyzed with an anti-Tie1 ELISA and sequencing. 20 HC were rescued with the whole LC diversity (phage DY3F85), and purified. Phage solution was blocked in MPBST (0.1% TWEEN® 20 & 2% skim milk). Blocked phage was depleted on beads coated with biotinylated anti-Fc and beads coated with Trail-Fc, for a total of 5 depletions, 10 minutes each. 200 pmol Tie-1-Fc was incubated with beads coated with bio-anti-Fc (500 μL total volume) O/N at 4° C. Depleted phage solution was added to target beads and incubated for 30 min at RT. Beads were washed 12× with PBST and beads with phage bound to them were used to infect 20 mL of HC-cells. Output was titered on Amp and Kan plates. ELISA 384 well plates were coated with Tie-1, anti-V5, anti-Kappa, anti Lambda or Trail-Fc (1 μg/mL, 100 μl/well in PBS), O/N at 4° C. The plates were blocked with 1% BSA in PBS, 1 hr at 37° C. and washed with PBST (0.1% TWEEN® 20). Supernatant was added to wells and incubated 1 hour at room temperature. Anti-M13-HRP was added and incubated lhr at room temperature. The plates were washed, substrate added, and read at 630 nm. For Plate #1, 34 isolates met the criteria T>0.5 & T/B>3. For Plate #2, 29 isolates met the criteria T>0.5 & T/B>3.

### EXAMPLE 2: VH/VL-CL Re-Linkage in the ROLIC method

This method is one way to allow re-establishment of the genotype linkage between the light chain and the heavy chain genes lost during the ROLIC cloning procedure (different ROLIC vectors for light chain and for heavy chain). It allows a one-step cloning of the antibody cassette back into pMID21 vector as ApalI-NheI fragment. If pMID21.03 is used as recipient, then we obtain a vector for production of sFabs. Briefly, the steps of the method are:

1. Infect HC bacteria with LC phage

2. PEG precipitate phage or just take the supernatant without PEG

3. Select for target binding

4. Collect bound phage - which only have LC DNA

5. Infect HC bacteria with LC phage

6. Plate for single colonies to keep LC and HC together - but not same pairs as in selection

7. Pick single colony in 96-well plate to allow screening by ELISA

8. Collect overnight phage supernatant and perform ELISA to check for binding to target

9. Use bacteria plate from step 7 (that still contain both HC-LC genes), amplify light chain and heavy chain separately and perform the zipping with RBS-like linker (see details on primers below)

10. Zipped antibody cassette is ready to be re-cloned into pMID21 as ApalI-NheI PCR insert

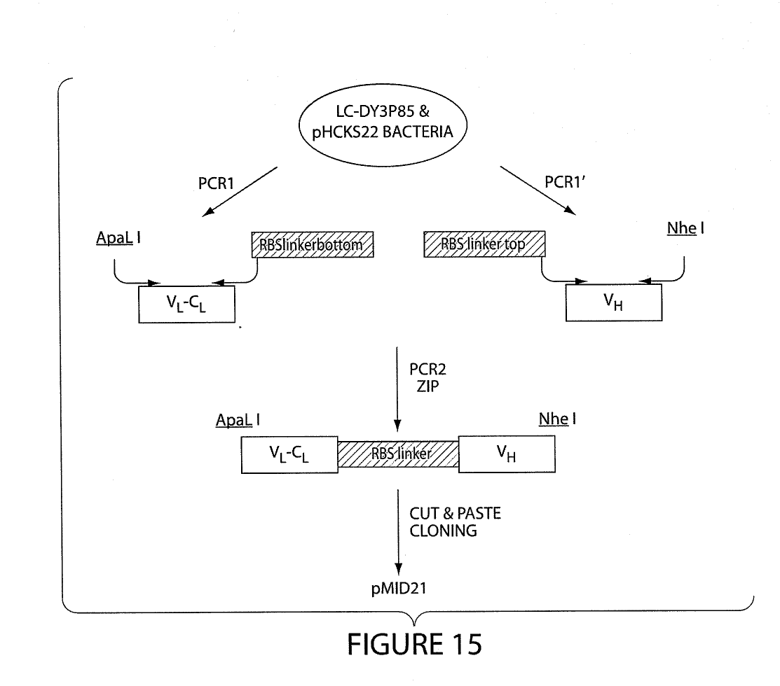

An overview of this method is shown in FIG. 15.

Primers to zip the light chain to the heavy chain and to allow a one-step cloning into the pMID21 vector:

1—Amplification of the heavy chain gene—appending RBS linker:

| RBS linker-HC top | | |
|---|---|---|
| | rbs-------------------HC leader----------- | |
| HCT1 | 5′-ggcgcgcctaaccatctatttcaaggagacagtcata Atgaagaagctcctctttgct-3′ | (SEQ ID NO:1) |
| HCT2 | 5′-ggcgcgcctaaccatctatttcaaggagacagtcata atgaaaaagcttttattcatg-3′ | (SEQ ID NO:2) |
| HCT3 | 5′-ggcgcgcctaaccatctatttcaagga ACAGTCTTA atgaaaaagcttttattcatg-3′ | (SEQ ID NO:3) |

The three primers are used together, as different members of the library may contain any one of the three sequences.

| | |
|---|---|
| HC bottom | |
| HCBot | |
| 5′-c tgggctgcct ggtcaaggac-3′ | (SEQ ID NO: 4) |

2—Amplification of the light chain gene—appending RBS linker:

LCss top
LCtop

(SEQ ID NO:5)

5′-cgcaattcctttagttgttc-3′
Lift LC AscI-RBS linker bottom
Kappa

(SEQ ID NO:6)

5′-AgcTTcAAcA ggggAgAgTg TTAATAAggc gcgccTAAcc
ATcTATTTcA AggAAcAgTcTTAA-3′
Lambda_bot2

(SEQ ID NO:7)

5′-cAgTggcccc TAcAgAATgT TcATAATAAg gcgcgccTAA
ccATcTATTT cAAggAgAcA gTcATA-3′
Lambda_Bot7

(SEQ ID NO:8)

5′-cAgTggcccc TgcAgAATgc TcTTAATAAg gcgcgccTAA
ccATcTATTT cAAggAgAcA gTcATA-3′

There are two primers for lambda because the library contains members with either Clambda 2 or Clambda 7.

3—Zipping step

| | |
|---|---|
| LC nested top | |
| 5′-gttcctttctattctcacagtg-3′ | (SEQ ID NO:9) |
| HC nested bottom | |
| 5′-gcAcccTccTccAAgAgcAc-3′ | (SEQ ID NO:10) |

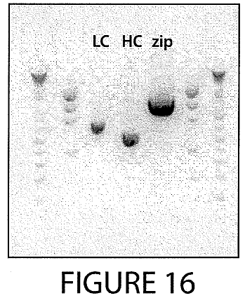

One clone was selected to demonstrate the concept of zipping, optimized as a 1-step reaction. FIG. 16 depicts an SDS-PAGE of the zipped construct compared to LC and HC alone.

### EXAMPLE 3: Economical Selection of Heavy Chains (ESCH)

It has often been noted that much of the affinity and specificity of antibodies derives from the HC and that LCs need only be permissive. Thus, it is possible to reverse the roles in ROLIC as described in Example 1: place a small population of LC in a vector that causes them to be secreted and build a new library of HCs in phage. These can then be combined by the much more efficient method of infection. Once a small set of effective HC are selected, these can be fed into ROLIC to obtain an optimal HC/LC pairing or they could be used as is.

One aspect of picking antibodies for use as human therapeutics is that we wish to avoid departures from germline sequence that are not essential to impart the desired affinity, specificity, solubility, and stability of the antibody. Thus, antibodies selected from phage libraries, from mice, or from humanized mice must be “germlined”. That is, all framework residues that are not germline are reverted to germline and the effect on the properties of the antibody examined, which is a lot of work. Hence, a highly useful approach would be to make a library of LC in cells where all the LCs have framework regions that are fully germlined. For example, we could select from an existing library for a set of LC that have fully germlined frameworks and some diversity, especially in LC-CDR3. The vector pLCSK24 is like pHCSK22 except that it is prepared to accept LC genes and to cause their secretion into the periplasm. DY3F87HC is like DY3F85LC except that it is arranged to accept VH-CH1 genes and to display them attached to IIIₛₜᵤₘₚ.

### EXAMPLE 4: Use of ROLIC for Affinity Maturation

We used the ROLIC method as an affinity maturation method for 6 antibody inhibitors of plasma kallikrein (pKal). Briefly, the method provides a means of allowing the 6 HC of these antibodies to be tested with our entire LC repertoire.

Six heavy chains were selected based on inhibition criteria and species cross reactivity studies to be matured using the ROLIC method. The 6 heavy chains were cloned into the pHCSK22 expression vector and TG1 cells were transformed with the plasmids. The bacteria were then infected with the light chain-containing phage which had been created by cloning the light chain repertoire into the DY3F85LC vector. Phage were assembled containing light chain fused to domain 3-transmembrane-intracellular anchor of the protein coded for by M13 geneIlI so that LC is anchored to the phage. These phage contain no HC component. HC protein is provided by the cellular HC library.

Other phage were constructed in which HC is fused to domain 3-transmembrane-intracellular anchor of the protein coded for by M13 geneIIlI so that HC is anchored to the phage. These phage contain not LC component. LC protein will be provided by a cellular LC library. Selections were performed using biotinylated human pKal protein on streptavidin magnetic beads or biotinylated mouse pKal protein on streptavidin magnetic beads as follows:

I. Human only

a. Round 1: 200 pmol human protein

b. Round 2: 100 pmol human protein

II. Mouse only

a. Round 1: 200 pmol mouse protein

b. Round 2: 100 pmol mouse protein

III. Human and mouse

a. Round 1: 200 pmol human protein

b. Round 2: 100 pmol mouse protein

Fresh TG1 cells containing the 6 heavy chains in pHCSK22 were infected with the resulting phage outputs between rounds. The phage were amplified overnight and used for the subsequent round of selection. At the end of round 2, new TG1 cells containing the 6 heavy chains were infected with the phage outputs and plated for growth of single colonies. The separate colonies were amplified in liquid growth in 96-well plates overnight and the supernatants containing the phage were tested for binding to biotinylated human and mouse pKal by standard ELISA.

A total of 672 colonies were tested by ELISA and 136 clones bound to both mouse and human pKal. There were some isolates that bound to mouse pKal only and others that bound to human pKal only. The light chains and heavy chains of these 136 dual binding isolates were PCR amplified individually, zipped together into single DNA strand via overlapping PCR oligos, and cloned into the pMID21 sFab expression vector (no geneIII). Sequence analysis resulted in 148 unique light chains paired to 3 of the 6 original heavy chains. Some mutations occurred in the PCR, inflating the number of LC-HC pairs.

### Example 5: Alternative primers for zipping LC and HC together

Below is an additional example of reagents and methods that can be used to re-link LC and HC together.

Heavy chains will come from pHCSK22 vector

All heavy chains will contain the hybrid7 signal sequence due to pHCSK22 vector construction

Actual hybrid7 signal sequence:

(SEQ ID NO:11)

ATGAAGAAGC TCCTCTTTGC TATCCCGCTC GTCGTTCCTT
TTGTGGCCCA GCCGGCCATG GCC

Light chains will come from DY3F85LC phage vector

No stop codons in the DY3F85LC vector thus they will need to be built back in addition to the RBS

The RBS sequence will be built back based on the actual sequence contained in the pMID21 vector stock as noted in the vector full sequence

Lambda constant region oligos are based on germline and webphage thus the C0 primer

The sequence between the last codon of LC and the first codon of HC SS is

(SEQ ID NO:12)

5′-taataaGGCGCGCCtaaccatctatttcaaggaacagtctta-3′

Theoretical constructs have been built containing a kappa or a hypothetical lambda using the hybrid7 and actual RBS
pMID21 kappa zip sample from ROLIC
pMiD21 lambda zip sample from ROLIC

Optional step: lift the light chains and heavy chains without lengthy tails prior to zipping, resulting in 3 PCR events total

All oligonucleotide (ON) sequences are in Table 1 below

Method:
PCR from LCss (ApaLI) to LCconst
   G3ss.For and
   Kconst Rev and
   Lambda C0 Rev and
   Lambda C2 Rev and
   Lambda C3 Rev and
   Lambda C7 Rev
PCR from HCss to NheI site
   HCss.For and
   HC.const.rev.
PCR from LCss (ApaLI) to LC+RBS overhang
   G3ss.For and
   K.RBS.Rev or
   LCO.RBS.Rev
   LC2.RBS.Rev
   LC3.RBS.Rev
   LC7.RBS.Rev
PCR from RBS+HCss to HCconst (NheI site)
   HCss.RBS.For and
   HC.const.rev
Zip from LCss (ApaLI) to HC const (NheI site)
   G3ss.For and
   HC.const.rev
Clone into pMID21 via ApaLI to NheI

TABLE 1

| | | |
|---|---|---|
| ON name | Sequence (5′-to-3′) | Use |

| | | |
|---|---|---|
| G3ss.For | CCTTTAGTTG TTCCTTTCTA | PCR LC, top |
| | TTCTCACAGT GCA | strand |
| | (SEQ ID NO:13) | |
| HC_const_Rev | GGAGGAGGGT GCTAGCGGGA | PCR HC, bottom |
| | AGACC | strand |
| | (SEQ ID NO:14) | |
| HCss For | ATGAAGAAGC TCCTCTTTGC | PCR HC, top |
| | T | strand |
| | (SEQ ID NO:15) | |
| HCss_RBS_For | CTAACCATCT ATTTCAAGGA | PCR HC signal |
| | ACAGTCTTAA TGAAGAAGCT | sequence, top |
| | CCTCTTTGCT | strand |
| | (SEQ ID NO:16) | |
| K_RBS_Rev | TTGAAATAGA TGGTTAGGCG | PCR kappa from |
| | CGCCTTATTA ACACTCTCCC | RBS |
| | CTGTTGAAG | |
| | (SEQ ID NO:17) | |
| Kconst Rev | ACACTCTCCC CTGTTGAAGC | PCR kappa, |
| | TCTT | lower strand |
| | (SEQ ID NO:18) | |
| Lambda C0 Rev | TGAACATTCT GTAGGGGCTA | PCR lambda, |
| | CTGTC | lower strand |
| | (SEQ ID NO:19) | |
| Lambda C2 Rev | TGAACATTCT GTAGGGGCCA | PCR lambda, |
| | CTGTC | lower strand |
| | (SEQ ID NO:20) | |
| Lambda C3 Rev | TGAACATTCC GTAGGGGCAA | PCR lambda, |
| | CTGTC | lower strand |
| | (SEQ ID NO:21) | |
| Lambda C7 Rev | AGAGCATTCT GCAGGGGCCA | PCR lambda, |
| | CTGTC | lower strand |
| | (SEQ ID NO:22) | |
| LC0_RBS For | TTGAAATAGA TGGTTAGGCG | PCR lambda from |
| | CGCCTTATTA TGAACATTCT | RBS to AscI |
| | GTAGGGGCTA | site, lower |
| | (SEQ ID NO:23) | strand |
| LC2_RBS For | TTGAAATAGA TGGTTAGGCG | PCR lambda from |
| | CGCCTTATTA TGAACATTCT | RBS to AscI |
| | GTAGGGGCC | site, lower |
| | (SEQ ID NO:24) | strand |
| LC3_RBS For | TTGAAATAGA TGGTTAGGCG | PCR lambda from |
| | CGCCTTATTA TGAACATTCC | RBS to AscI |
| | GTAGGGGCAA | site, lower |
| | (SEQ ID NO:25) | strand |
| LC7_RBS For | TTGAAATAGA TGGTTAGGCG | PCR lambda from |
| | CGCCTTATTA AGAGCATTCT | RBS to AscI |
| | GCAGGGGCC | site, lower |
| | (SEQ ID NO:26) | strand |

TABLE 2

The DNA sequence of DY3F85LC containing a sample germline O12 kappa light
chain. The antibody sequences shown are of the form of actual antibody,
but have not been identified as binding to a particular antigen.
On each line, everything after an exclamation point (!) is commentary.
The DNA of DY3F85LC is (SEQ ID NO: 27)

!----------------------------------------------------------------------------

| | | |
|---|---|---|
| | 1 | AATGCTACTA CTATTAGTAG AATTGATGCC ACCTTTTCAG CTCGCGCCCC AAATGAAAAT |
| | 61 | ATAGCTAAAC AGGTTATTGA CCATTTGCGA AATGTATCTA ATGGTCAAAC TAAATCTACT |
| | 121 | CGTTCGCAGA ATTGGGAATC AACTGTTATA TGGAATGAAA CTTCCAGACA CCGTACTTTA |
| | 181 | GTTGCATATT TAAAACATGT TGAGCTACAG CATTATATTC AGCAATTAAG CTCTAAGCCA |
| | 241 | TCCGCAAAAA TGACCTCTTA TCAAAAGGAG CAATTAAAGG TACTCTCTAA TCCTGACCTG |
| | 301 | TTGGAGTTTG CTTCCGGTCT GGTTCGCTTT GAAGCTCGAA TTAAAACGCG ATATTTGAAG |
| | 361 | TCTTTCGGGC TTCCTCTTAA TCTTTTTGAT GCAATCCGCT TTGCTTCTGA CTATAATAGT |
| | 421 | CAGGGTAAAG ACCTGATTTT TGATTTATGG TCATTCTCGT TTTCTGAACT GTTTAAAGCA |
| | 481 | TTTGAGGGGG ATTCAATGAA TATTTATGAC GATTCCGCAG TATTGGACGC TATCCAGTCT |
| | 541 | AAACATTTTA CTATTACCCC CTCTGGCAAA ACTTCTTTTG CAAAAGCCTC TCGCTATTTT |
| | 601 | GGTTTTTATC GTCGTCTGGT AAACGAGGGT TATGATAGTG TTGCTCTTAC TATGCCTCGT |
| | 661 | AATTCCTTTT GGCGTTATGT ATCTGCATTA GTTGAATGTG GTATTCCTAA ATCTCAACTG |
| | 721 | ATGAATCTTT CTACCTGTAA TAATGTTGTT CCGTTAGTTC GTTTTATTAA CGTAGATTTT |
| | 781 | TCTTCCCAAC GTCCTGACTG GTATAATGAG CCAGTTCTTA AAATCGCATA AGGTAATTCA |
| | 841 | CAATGATTAA AGTTGAAATT AAACCATCTC AAGCCCAATT TACTACTCGT TCTGGTGTTT |
| | 901 | CTCGTCAGGG CAAGCCTTAT TCACTGAATG AGCAGCTTTG TTACGTTGAT TTGGGTAATG |
| | 961 | AATATCCGGT TCTTGTCAAG ATTACTCTTG ATGAAGGTCA GCCAGCCTAT GCGCCTGGTC |
| | 1021 | TGTACACCGT TCATCTGTCC TCTTTCAAAG TTGGTCAGTT CGGTTCCCTT ATGATTGACC |
| | 1081 | GTCTGCGCCT CGTTCCGGCT AAGTAACATG GAGCAGGTCG CGGATTTCGA CACAATTTAT |
| | 1141 | CAGGCGATGA TACAAATCTC CGTTGTACTT TGTTTCGCGC TTGGTATAAT CGCTGGGGGT |
| | 1201 | CAAAGATGAG TGTTTTAGTG TATTCTTTTG CCTCTTTCGT TTTAGGTTGG TGCCTTCGTA |
| | 1261 | GTGGCATTAC GTATTTTACC CGTTTAATGG AAACTTCCTC ATGAAAAAGT CTTTAGTCCT |
| | 1321 | CAAAGCCTCT GTAGCCGTTG CTACCCTCGT TCCGATGCTG TCTTTCGCTG CTGAGGGTGA |
| | 1381 | CGATCCCGCA AAAGCGGCCT TTAACTCCCT GCAAGCCTCA GCGACCGAAT ATATCGGTTA |
| | 1441 | TGCGTGGGCG ATGGTTGTTG TCATTGTCGG CGCAACTATC GGTATCAAGC TGTTTAAGAA |
| | 1501 | ATTCACCTCG AAAGCAAGCT GATAAACCGA TACAATTAAA GGCTCCTTTT GGAGCCTTTT |
| | 1561 | TTTTGGAGAT TTTCAACGTG AAAAAATTAT TATTCGCAAT TCCTTTAGTT GTTCCTTTCT |
| | 1621 | ATTCTCACTC CGCTGAAACT GTTGAAAGTT GTTTAGCAAA ATCCCATACA GAAAATTCAT |
| | 1681 | TTACTAACGT CTGGAAAGAC GACAAAACTT TAGATCGTTA CGCTAACTAT GAGGGCTGTC |
| | 1741 | TGTGGAATGC TACAGGCGTT GTAGTTTGTA CTGGTGACGA AACTCAGTGT TACGGTACAT |
| | 1801 | GGGTTCCTAT TGGGCTTGCT ATCCCTGAAA ATGAGGGTGG TGGCTCTGAG GGTGGCGGTT |
| | 1861 | CTGAGGGTGG CGGTTCTGAG GGTGGCGGTA CTAAACCTCC TGAGTACGGT GATACACCTA |
| | 1921 | TTCCGGGCTA TACTTATATC AACCCTCTCG ACGGCACTTA TCCGCCTGGT ACTGAGCAAA |
| | 1981 | ACCCCGCTAA TCCTAATCCT TCTCTTGAGG AGTCTCAGCC TCTTAATACT TTCATGTTTC |
| | 2041 | AGAATAATAG GTTCCGAAAT AGGCAGGGGG CATTAACTGT TTATACGGGC ACTGTTACTC |
| | 2101 | AAGGCACTGA CCCCGTTAAA ACTTATTACC AGTACACTCC TGTATCATCA AAAGCCATGT |
| | 2161 | ATGACGCTTA CTGGAACGGT AAATTCAGAG ACTGCGCTTT CCATTCTGGC TTTAATGAGG |
| | 2221 | ATTTATTTGT TTGTGAATAT CAAGGCCAAT CGTCTGACCT GCCTCAACCT CCTGTCAATG |
| | 2281 | CTGGCGGCGG CTCTGGTGGT GGTTCTGGTG GCGGCTCTGA GGGTGGTGGC TCTGAGGGTG |
| | 2341 | GCGGTTCTGA GGGTGGCGGC TCTGAGGGAG GCGGTTCCGG TGGTGGCTCT GGTTCCGGTG |
| | 2401 | ATTTTGATTA TGAAAAGATG GCAAACGCTA ATAAGGGGGC TATGACCGAA AATGCCGATG |
| | 2461 | AAAACGCGCT ACAGTCTGAC GCTAAAGGCA AACTTGATTC TGTCGCTACT GATTACGGTG |
| | 2521 | CTGCTATCGA TGGTTTCATT GGTGACGTTT CCGGCCTTGC TAATGGTAAT GGTGCTACTG |
| | 2581 | GTGATTTTGC TGGCTCTAAT TCCCAAATGG CTCAAGTCGG TGACGGTGAT AATTCACCTT |
| | 2641 | TAATGAATAA TTTCCGTCAA TATTTACCTT CCCTCCCTCA ATCGGTTGAA TGTCGCCCTT |
| | 2701 | TTGTCTTTGG CGCTGGTAAA CCATATGAAT TTTCTATTGA TTGTGACAAA ATAAACTTAT |
| | 2761 | TCCGTGGTGT CTTTGCGTTT CTTTTATATG TTGCCACCTT TATGTATGTA TTTTCTACGT |
| | 2821 | TTGCTAACAT ACTGCGTAAT AAGGAGTCTT AATCATGCCA GTTCTTTTGG GTATTCCGTT |
| | 2881 | ATTATTGCGT TTCCTCGGTT TCCTTCTGGT AACTTTGTTC GGCTATCTGC TTACTTTTCT |
| | 2941 | TAAAAAGGGC TTCGGTAAGA TAGCTATTGC TATTTCATTG TTTCTTGCTC TTATTATTGG |
| | 3001 | GCTTAACTCA ATTCTTGTGG GTTATCTCTC TGATATTAGC GCTCAATTAC CCTCTGACTT |
| | 3061 | TGTTCAGGGT GTTCAGTTAA TTCTCCCGTC TAATGCGCTT CCCTGTTTTT ATGTTATTCT |
| | 3121 | CTCTGTAAAG GCTGCTATTT TCATTTTTGA CGTTAAACAA AAAATCGTTT CTTATTTGGA |
| | 3181 | TTGGGATAAA TAATATGGCT GTTTATTTTG TAACTGGCAA ATTAGGCTCT GGAAAGACGC |
| | 3241 | TCGTTAGCGT TGGTAAGATT CAGGATAAAA TTGTAGCTGG GTGCAAAATA GCAACTAATC |
| | 3301 | TTGATTTAAG GCTTCAAAAC CTCCCGCAAG TCGGGAGGTT CGCTAAAACG CCTCGCGTTC |
| | 3361 | TTAGAATACC GGATAAGCCT TCTATATCTG ATTTGCTTGC TATTGGGCGC GGTAATGATT |
| | 3421 | CCTACGATGA AAATAAAAAC GGCTTGCTTG TTCTCGATGA GTGCGGTACT TGGTTTAATA |
| | 3481 | CCCGTTCTTG GAATGATAAG GAAAGACAGC CGATTATTGA TTGGTTTCTA CATGCTCGTA |
| | 3541 | AATTAGGATG GGATATTATT TTTCTTGTTC AGGACTTATC TATTGTTGAT AAACAGGCGC |
| | 3601 | GTTCTGCATT AGCTGAACAT GTTGTTTATT GTCGTCGTCT GGACAGAATT ACTTTACCTT |
| | 3661 | TTGTCGGTAC TTTATATTCT CTTATTACTG GCTCGAAAAT GCCTCTGCCT AAATTACATG |
| | 3721 | TTGGCGTTGT TAAATATGGC GATTCTCAAT TAAGCCCTAC TGTTGAGCGT TGGCTTTATA |
| | 3781 | CTGGTAAGAA TTTGTATAAC GCATATGATA CTAAACAGGC TTTTTCTAGT AATTATGATT |
| | 3841 | CCGGTGTTTA TTCTTATTTA ACGCCTTATT TATCACACGG TCGGTATTTC AAACCATTAA |
| | 3901 | ATTTAGGTCA GAAGATGAAA TTAACTAAAA TATATTTGAA AAAGTTTTCT CGCGTTCTTT |
| | 3961 | GTCTTGCGAT TGGATTTGCA TCAGCATTTA CATATAGTTA TATAACCCAA CCTAAGCCGG |
| | 4021 | AGGTTAAAAA GGTAGTCTCT CAGACCTATG ATTTTGATAA ATTCACTATT GACTCTTCTC |
| | 4081 | AGCGTCTTAA TCTAAGCTAT CGCTATGTTT TCAAGGATTC TAAGGGAAAA TTAATTAATA |
| | 4141 | GCGACGATTT ACAGAAGCAA GGTTATTCAC TCACATATAT TGATTTATGT ACTGTTTCCA |
| | 4201 | TTAAAAAAGG TAATTCAAAT GAAATTGTTA AATGTAATTA ATTTTGTTTT CTTGATGTTT |
| | 4261 | GTTTCATCAT CTTCTTTTGC TCAGGTAATT GAAATGAATA ATTCGCCTCT GCGCGATTTT |
| | 4321 | GTAACTTGGT ATTCAAAGCA ATCAGGCGAA TCCGTTATTG TTTCTCCCGA TGTAAAAGGT |
| | 4381 | ACTGTTACTG TATATTCATC TGACGTTAAA CCTGAAAATC TACGCAATTT CTTTATTTCT |
| | 4441 | GTTTTACGTG CAAATAATTT TGATATGGTA GGTTCTAACC CTTCCATAAT TCAGAAGTAT |
| | 4501 | AATCCAAACA ATCAGGATTA TATTGATGAA TTGCCATCAT CTGATAATCA GGAATATGAT |
| | 4561 | GATAATTCCG CTCCTTCTGG TGGTTTCTTT GTTCCGCAAA ATGATAATGT TACTCAAACT |
| | 4621 | TTTAAAATTA ATAACGTTCG GGCAAAGGAT TTAATACGAG TTGTCGAATT GTTTGTAAAG |
| | 4681 | TCTAATACTT CTAAATCCTC AAATGTATTA TCTATTGACG GCTCTAATCT ATTAGTTGTT |
| | 4741 | AGTGCTCCTA AAGATATTTT AGATAACCTT CCTCAATTCC TTTCAACTGT TGATTTGCCA |
| | 4801 | ACTGACCAGA TATTGATTGA GGGTTTGATA TTTGAGGTTC AGCAAGGTGA TGCTTTAGAT |
| | 4861 | TTTTCATTTG CTGCTGGCTC TCAGCGTGGC ACTGTTGCAG GCGGTGTTAA TACTGACCGC |
| | 4921 | CTCACCTCTG TTTTATCTTC TGCTGGTGGT TCGTTCGGTA TTTTTAATGG CGATGTTTTA |
| | 4981 | GGGCTATCAG TTCGCGCATT AAAGACTAAT AGCCATTCAA AAATATTGTC TGTGCCACGT |
| | 5041 | ATTCTTACGC TTTCAGGTCA GAAGGGTTCT ATCTCTGTTG GCCAGAATGT CCCTTTTATT |
| | 5101 | ACTGGTCGTG TGACTGGTGA ATCTGCCAAT GTAAATAATC CATTTCAGAC GATTGAGCGT |
| | 5161 | CAAAATGTAG GTATTTCCAT GAGCGTTTTT CCTGTTGCAA TGGCTGGCGG TAATATTGTT |
| | 5221 | CTGGATATTA CCAGCAAGGC CGATAGTTTG AGTTCTTCTA CTCAGGCAAG TGATGTTATT |
| | 5281 | ACTAATCAAA GAAGTATTGC TACAACGGTT AATTTGCGTG ATGGACAGAC TCTTTTACTC |
| | 5341 | GGTGGCCTCA CTGATTATAA AAACACTTCT CAGGATTCTG GCGTACCGTT CCTGTCTAAA |
| | 5401 | ATCCCTTTAA TCGGCCTCCT GTTTAGCTCC CGCTCTGATT CTAACGAGGA AAGCACGTTA |
| | 5461 | TACGTGCTCG TCAAAGCAAC CATAGTACGC GCCCTGTAGC GGCGCATTAA GCGCGGCGGG |
| | 5521 | TGTGGTGGTT ACGCGCAGCG TGACCGCTAC ACTTGCCAGC GCCCTAGCGC CCGCTCCTTT |
| | 5581 | CGCTTTCTTC CCTTCCTTTC TCGCCACGTT CGCCGGCTTT CCCCGTCAAG CTCTAAATCG |
| | 5641 | GGGGCTCCCT TTAGGGTTCC GATTTAGTGC TTTACGGCAC CTCGACCCCA AAAAACTTGA |
| | 5701 | TTTGGGTGAT GGTTCACGTA GTGGGCCATC GCCCTGATAG ACGGTTTTTC GCCCTTTGAC |
| | 5761 | GTTGGAGTCC ACGTTCTTTA ATAGTGGACT CTTGTTCCAA ACTGGAACAA CACTCAACCC |
| | 5821 | TATCTCGGGC TATTCTTTTG ATTTATAAGG GATTTTGCCG ATTTCGGAAC CACCATCAAA |
| | 5881 | CAGGATTTTC GCCTGCTGGG GCAAACCAGC GTGGACCGCT TGCTGCAACT CTCTCAGGGC |
| | 5941 | CAGGCGGTGA AGGGCAATCA GCTGTTGCCC GTCTCACTGG TGAAAAGAAA AACCACCCTG |
| | 6001 | GATCCAAGCT TGCAGGTGGC ACTTTTCGGG GAAATGTGCG CGGAACCCCT ATTTGTTTAT |
| | 6061 | TTTTCTAAAT ACATTCAAAT ATGTATCCGC TCATGAGACA ATAACCCTGA TAAATGCTTC |
| | 6121 | AATAATATTG AAAAAGGAAG AGTATGAGTA TTCAACATTT CCGTGTCGCC CTTATTCCCT |
| | 6181 | TTTTTGCGGC ATTTTGCCTT CCTGTTTTTG CTCACCCAGA AACGCTGGTG AAAGTAAAAG |
| | 6241 | ATGCTGAAGA TCAGTTGGGC GCACTAGTGG GTTACATCGA ACTGGATCTC AACAGCGGTA |
| | 6301 | AGATCCTTGA GAGTTTTCGC CCCGAAGAAC GTTTTCCAAT GATGAGCACT TTTAAAGTTC |
| | 6361 | TGCTATGTGG CGCGGTATTA TCCCGTATTG ACGCCGGGCA AGAGCAACTC GGTCGCCGCA |
| | 6421 | TACACTATTC TCAGAATGAC TTGGTTGAGT ACTCACCAGT CACAGAAAAG CATCTTACGG |
| | 6481 | ATGGCATGAC AGTAAGAGAA TTATGCAGTG CTGCCATAAC CATGAGTGAT AACACTGCGG |
| | 6541 | CCAACTTACT TCTGACAACG ATCGGAGGAC CGAAGGAGCT AACCGCTTTT TTGCACAACA |
| | 6601 | TGGGGGATCA TGTAACTCGC CTTGATCGTT GGGAACCGGA GCTGAATGAA GCCATACCAA |
| | 6661 | ACGACGAGCG TGACACCACG ATGCCTGTAG CAATGGCAAC AACGTTGCGC AAACTATTAA |
| | 6721 | CTGGCGAACT ACTTACTCTA GCTTCCCGGC AACAATTAAT AGACTGGATG GAGGCGGATA |
| | 6781 | AAGTTGCAGG ACCACTTCTG CGCTCGGCCC TTCCGGCTGG CTGGTTTATT GCTGATAAAT |
| | 6841 | CTGGAGCCGG TGAGCGTGGG TCTCGCGGTA TCATTGCAGC ACTGGGGCCA GATGGTAAGC |
| | 6901 | CCTCCCGTAT CGTAGTTATC TACACGACGG GGAGTCAGGC AACTATGGAT GAACGAAATA |
| | 6961 | GACAGATCGC TGAGATAGGT GCCTCACTGA TTAAGCATTG GTAACTGTCA GACCAAGTTT |
| | 7021 | ACTCATATAT ACTTTAGATT GATTTAAAAC TTCATTTTTA ATTTAAAAGG ATCTAGGTGA |
| | 7081 | AGATCCTTTT TGATAATCTC ATGACCAAAA TCCCTTAACG TGAGTTTTCG TTCCACTGTA |
| | 7141 | CGTAAGACCC CCAAGCTTGT CGACTGAATG GCGAATGGCG CTTTGCCTGG TTTCCGGCAC |
| | 7201 | CAGAAGCGGT GCCGGAAAGC TGGCTGGAGT GCGATCTTCC TGACGCTCGA GCGCAACGCA |
| ! | XhoI . . . | |
| | 7261 | ATTAATGTGA GTTAGCTCAC TCATTAGGCA CCCCAGGCTT TACACTTTAT GCTTCCGGCT |
| | 7321 | CGTATGTTGT GTGGAATTGT GAGCGGATAA CAATTTCACA CAGGAAACAG CTATGACCAT |
| | 7381 | GATTACGCCA AGCTTTGGAG CCTTTTTTTT GGAGATTTTC AAC |
| ! | | |

| | |
|---|---|
| ! | The polypeptide encoded by bases 7424-8673 are (SEQ ID NO: 28) |

| | | |
|---|---|---|
| ! | | Signal sequence------------------------------------------- |
| ! | | 1 2 3 4 5 6 7 8 9 10 11 12 13 14 15 |
| ! | | M K K L L F A I P L V V P F Y |
| | 7424 | gtg aaa aaa tta tta ttc gca att cct tta gtt gtt cct ttc tat |
| ! | | |
| ! | | Signal . . . Kappa O12 Vlight-------- FR1 --------- |
| ! | | 16 17 18 19 20 21 22 23 24 25 26 27 28 29 30 |
| ! | | S H S A Q D I Q M T Q S P S S |
| | 7469 | tct cac aGT GCA Caa gac atc cag atg acc cag tct cca tcc tcc |
| ! | | ApaLI . . . |
| ! | | |
| ! | | FR1 ---------------------------------------------- CDR1--- |
| ! | | 31 32 33 34 35 36 37 38 39 40 41 42 43 44 45 |
| ! | | L S A S V G D R V T I T C R A |
| | 7514 | ctg tct gct tct gtt ggg gat aga gtc acc atc acc tgc agg gcc |
| ! | | |
| ! | | CDR1------------------------------- FR2------------------- |
| ! | | 46 47 48 49 50 51 52 53 54 55 56 57 58 59 60 |
| ! | | S Q S I S S Y L N W Y Q Q K P |
| | 7559 | agt cag agt atc agc agc tat cta aat tGG TAC Caa cag aaa cct |
| ! | | KpnI . . . |
| ! | | |
| ! | | FR2------------------------------- CDR2------------------ |
| ! | | 61 62 63 64 65 66 67 68 69 70 71 72 73 74 75 |
| ! | | G K A P K L L I Y A A S S L Q |
| | 7604 | ggc aag gct ccc aag ctc ctc atc tat gct gca tcc tct ttg caa |
| ! | | |
| ! | | CDR2 FR3--------------------------------------------------- |
| ! | | 76 77 78 79 80 81 82 83 84 85 86 87 88 89 90 |
| ! | | S G V P S R F S G S G S G T D |
| | 7649 | tca ggc gtc cca agc agg ttc agt ggc agt ggg tct ggg aca gac |
| ! | | |
| ! | | FR3 ------------------------------------------------------- |
| ! | | 91 92 93 94 95 96 97 98 99 100 101 102 103 104 105 |
| ! | | F I L T I S S L Q P E D F A T |
| | 7694 | ttc act ctc acc atc agc agt ctg cag cct gaa gat ttt gca acg |
| ! | | |
| ! | | FR3 ------- CDR3------------------------------- FR4-------- |
| ! | | 106 107 108 109 110 111 112 113 114 115 116 117 118 119 120 |
| ! | | Y Y C Q Q S Y S T P F T F G P |
| | 7739 | tat tac tgt caa cag tct tat agt aca cca ttc act ttc ggc cct |
| ! | | |
| ! | | FR4------------------------ Ckappa------------------------- |
| ! | | 121 122 123 124 125 126 127 128 129 130 131 132 133 134 135 |
| ! | | G T K V D I K R T V A A P S V |
| | 7784 | ggg acc aaa gtg gat atc aaa cga act gtg gct gca cca tct gtc |
| ! | | |
| ! | | Ckappa----------------------------------------------------- |
| ! | | 136 137 138 139 140 141 142 143 144 145 146 147 148 149 150 |
| ! | | F I F P P S D E Q L K S G T A |
| | 7829 | ttc atc ttc ccg cca tct gat gag cag ttg aaa tct gga act gcc |
| ! | | |
| ! | | Ckappa----------------------------------------------------- |
| ! | | 151 152 153 154 155 156 157 158 159 160 161 162 163 164 165 |
| ! | | S V V C L L N N F Y P R E A K |
| | 7874 | tct gtt gtg tgc ctg ctg aat aac ttc tat ccc aga gag gcc aaa |
| ! | | |
| ! | | Ckappa----------------------------------------------------- |
| ! | | 166 167 168 169 170 171 172 173 174 175 176 177 178 179 180 |
| ! | | V Q W K V D N A L Q S G N S Q |
| | 7919 | gta cag tgg aag gtg gat aac gcc ctc caa tcg ggt aac tcc cag |
| ! | | |
| ! | | Ckappa----------------------------------------------------- |
| ! | | 181 182 183 184 185 186 187 188 189 190 191 192 193 194 195 |
| ! | | E S V T E Q D S K D S T Y S L |
| | 7964 | gag agt gtc aca gag cag gac agc aag gac agc acc tac agc ctc |
| ! | | |
| ! | | Ckappa----------------------------------------------------- |
| ! | | 196 197 198 199 200 201 202 203 204 205 206 207 208 209 210 |
| ! | | S S T L T L S K A D Y E K H K |
| | 8009 | agc agc acc ctg acg ctg agc aaa gca gac tac gag aaa cac aaa |
| ! | | |
| ! | | Ckappa----------------------------------------------------- |
| ! | | 211 212 213 214 215 216 217 218 219 220 221 222 223 224 225 |
| ! | | V Y A C E V T H Q G L S S P V |
| | 8054 | gtc tac gcc tgc gaa gtc acc cat cag ggc ctG AGC TCg ccc gtc |
| ! | | SacI . . . |
| ! | | |
| ! | | Ckappa----------------------------- His tag---- |
| ! | | 226 227 228 229 230 231 232 233 234 235 236 237 238 239 240 |
| ! | | T K S F N R G E C A A A H H H |
| | 8099 | aca aag agc ttc aac agg gga gag tgt gcg gcc gca cat cat cat |
| ! | | NotI . . . |
| ! | | |
| ! | | His tag Myc tag---> |
| ! | | 241 242 243 244 245 246 247 248 249 250 251 252 253 254 255 |
| ! | | H H H G A A E Q K L I S E E D |
| | 8144 | cac cat cac ggg gcc gca gaa caa aaa ctc atc tca gaa gag gat |
| ! | | |
| ! | | Domain 3 of III . . . |
| ! | | 256 257 258 259 260 261 262 263 264 265 266 267 268 269 270 |
| ! | | L N G A A E A S S A S G D F D |
| | 8189 | ctg aat ggg gcc gca gag GCT AGC tct gct agt ggc gac ttc gac |
| ! | | NheI . . . |
| ! | | |
| ! | | Domain 3 of III-------------------------------------------- |
| ! | | 271 272 273 274 275 276 277 278 279 280 281 282 283 284 285 |
| ! | | |
| ! | | Domain 3 of III-------------------------------------------- |
| ! | | 286 287 288 289 290 291 292 293 294 295 296 297 298 299 300 |
| ! | | A D E N A L Q S D A K G K L D |
| | 8279 | gct gac gag aat gct ttg caa agc gat gcc aag ggt aag tta gac |
| ! | | |
| ! | | Domain 3 of III-------------------------------------------- |
| ! | | 301 302 303 304 305 306 307 308 309 310 311 312 313 314 315 |
| ! | | S V A T D Y G A A I D G F I G |
| | 8324 | agc gtc gcg acc gac tat ggc gcc gcc atc gac ggc ttt atc ggc |
| ! | | |
| ! | | 316 317 318 319 320 321 322 323 324 325 326 327 328 329 330 |
| ! | | D V S G L A N G N G A T G D F |
| | 8369 | gat gtc agt ggt ttg gcc aac ggc aac gga gcc acc gga gac ttc |
| ! | | |
| ! | | 331 332 333 334 335 336 337 338 339 340 341 342 343 344 345 |
| ! | | A G S N S Q M A Q V G D G D N |
| | 8414 | gca ggt tcg aat tct cag atg gcc cag gtt gga gat ggg gac aac |
| ! | | |
| ! | | 346 347 348 349 350 351 352 353 354 355 356 357 358 359 360 |
| ! | | S P L M N N F R Q Y L P S L P |
| | 8459 | agt ccg ctt atg aac aac ttt aga cag tac ctt ccg tct ctt ccg |
| ! | | |
| ! | | 361 362 363 364 365 366 367 368 369 370 371 372 373 374 375 |
| ! | | Q S V E C R P F V F G A G K P |
| | 8504 | cag agt gtc gag tgc cgt cca ttc gtt ttc ggt gcc ggc aag cct |
| ! | | |
| ! | | Transmem |
| ! | | 376 377 378 379 380 381 382 383 384 385 386 387 388 389 390 |
| ! | | Y E F S I D C D K I N L F R G |
| | 8549 | tac gag ttc agc atc gac tgc gat aag atc aat ctt ttc cgc ggc |
| ! | | |
| ! | | Transmembrane---------------------------------------------- |
| ! | | 391 392 393 394 395 396 397 398 399 400 401 402 403 404 405 |
| ! | | V F A F L L Y V A T F M Y V F |
| | 8594 | gtt ttc gct ttc ttg cta tac gtc gct act ttc atg tac gtt ttc |
| ! | | |
| ! | | Transmembrane-------------- Intracellular anchor |
| ! | | 406 407 408 409 410 411 412 413 414 415 416 417 418 419 |
| ! | | S T F A N I L R N K E S • • |
| | 8639 | agc act ttc gcc aat att tta cgc aac aaa gaa agc tag tga |
| ! | | |
| | 8681 | TCTCCTAGGA AGCCCGCCTA |
| | 8701 | ATGAGCGGGC TTTTTTTTTC TGGTATGCAT CCTGAGGCCG ATACTGTCGT CGTCCCCTCA |
| | 8761 | AACTGGCAGA TGCACGGTTA CGATGCGCCC ATCTACACCA ACGTGACCTA TCCCATTACG |
| | 8821 | GTCAATCCGC CGTTTGTTCC CACGGAGAAT CCGACGGGTT GTTACTCGCT CACATTTAAT |
| | 8881 | GTTGATGAAA GCTGGCTACA GGAAGGCCAG ACGCGAATTA TTTTTGATGG CGTTCCTATT |
| | 8941 | GGTTAAAAAA TGAGCTGATT TAACAAAAAT TTAATGCGAA TTTTAACAAA ATATTAACGT |
| | 9001 | TTACAATTTA AATATTTGCT TATACAATCT TCCTGTTTTT GGGGCTTTTC TGATTATCAA |
| | 9061 | CCGGGGTACA TATGATTGAC ATGCTAGTTT TACGATTACC GTTCATCGAT TCTCTTGTTT |
| | 9121 | GCTCCAGACT CTCAGGCAAT GACCTGATAG CCTTTGTAGA TCTCTCAAAA ATAGCTACCC |
| | 9181 | TCTCCGGCAT TAATTTATCA GCTAGAACGG TTGAATATCA TATTGATGGT GATTTGACTG |
| | 9241 | TCTCCGGCCT TTCTCACCCT TTTGAATCTT TACCTACACA TTACTCAGGC ATTGCATTTA |
| | 9301 | AAATATATGA GGGTTCTAAA AATTTTTATC CTTGCGTTGA AATAAAGGCT TCTCCCGCAA |
| | 9361 | AAGTATTACA GGGTCATAAT GTTTTTGGTA CAACCGATTT AGCTTTATGC TCTGAGGCTT |
| | 9421 | TATTGCTTAA TTTTGCTAAT TCTTTGCCTT GCCTGTATGA TTTATTGGAT GTT |

TABLE 3

Sequence of pHCSK22 with a representative sample HC. The antibody
sequences shown are of the form of actual antibody, but have not
been identified as binding to a particular antigen. On each line,
everything after an exclamation point (!) is commentary. The DNA
of pHCSK22 is SEQ ID NO: 29. The amino-acid sequence of the
polypeptide encoded by bases 2215-3021 is SEQ ID NO: 30.

| | |
|---|---|
| !pHCSK22 | 3457 CIRCULAR |

| | | |
|---|---|---|
| ! | | |
| | 1 | GACGAAAGGG CCTGCTCTGC CAGTGTTACA ACCAATTAAC CAATTCTGAT TAGAAAAACT |
| | 61 | CATCGAGCAT CAAATGAAAC TGCAATTTAT TCATATCAGG ATTATCAATA CCATATTTTT |
| | 121 | GAAAAAGCCG TTTCTGTAAT GAAGGAGAAA ACTCACCGAG GCAGTTCCAT AGGATGGCAA |
| | 181 | GATCCTGGTA TCGGTCTGCG ATTCCGACTC GTCCAACATC AATACAACCT ATTAATTTCC |
| | 241 | CCTCGTCAAA AATAAGGTTA TCAAGTGAGA AATCACCATG AGTGACGACT GAATCCGGTG |
| | 301 | AGAATGGCAA AAGCTTATGC ATTTCTTTCC AGACTTGTTC AACAGGCCAG CCATTACGCT |
| | 361 | CGTCATCAAA ATCACTCGCA TCAACCAAAC CGTTATTCAT TCGTGATTGC GCCTGAGCGA |
| | 421 | GACGAAATAC GCGATCGCTG TTAAAAGGAC AATTACAAAC AGGAATTGAA TGCAACCGGC |
| | 481 | GCAGGAACAC TGCCAGCGCA TCAACAATAT TTTCACCTGA ATCAGGATAT TCTTCTAATA |
| | 541 | CCTGGAATGC TGTTTTCCCG GGGATCGCAG TGGTGAGTAA CCATGCATCA TCAGGAGTAC |
| | 601 | GGATAAAATG CTTGATGGTC GGAAGAGGCA TAAATTCCGT CAGCCAGTTT AGTCTGACCA |
| | 661 | TCTCATCTGT AACATCATTG GCAACGCTAC CTTTGCCATG TTTCAGAAAC AACTCTGGCG |
| | 721 | CATCGGGCTT CCCATACAAT CGATAGATTG TCGCACCTGA TTGCCCGACA TTATCGCGAG |
| | 781 | CCCATTTATA CCCATATAAA TCAGCATCCA TGTTGGAATT TAATCGCGGC CTCGAGCAAG |
| | 841 | ACGTTTCCCG TTGAATATGG CTCATAACAC CCCTTGTATT ACTGTTTATG TAAGCAGACA |
| | 901 | GTTTTATTGT TCATGATGAT ATATTTTTAT CTTGTGCAAT GTAACATCAG AGATTTTGAG |
| | 961 | ACACAACGTG GCTTTCCCCC CCCCCCCCTG CAGGTCTCGG GCTATTCCTG TCAGACCAAG |
| | 1021 | TTTACTCATA TATACTTTAG ATTGATTTAA AACTTCATTT TTAATTTAAA AGGATCTAGG |
| | 1081 | TGAAGATCCT TTTTGATAAT CTCATGACCA AAATCCCTTA ACGTGAGTTT TCGTTCCACT |
| | 1141 | GAGCGTCAGA CCCCGTAGAA AAGATCAAAG GATCTTCTTG AGATCCTTTT TTTCTGCGCG |
| | 1201 | TAATCTGCTG CTTGCAAACA AAAAAACCAC CGCTACCAGC GGTGGTTTGT TTGCCGGATC |
| | 1261 | AAGAGCTACC AACTCTTTTT CCGAAGGTAA CTGGCTTCAG CAGAGCGCAG ATACCAAATA |
| | 1321 | CTGTTCTTCT AGTGTAGCCG TAGTTAGGCC ACCACTTCAA GAACTCTGTA GCACCGCCTA |
| | 1381 | CATACCTCGC TCTGCTAATC CTGTTACCAG TGGCTGCTGC CAGTGGCGAT AAGTCGTGTC |
| | 1441 | TTACCGGGTT GGACTCAAGA CGATAGTTAC CGGATAAGGC GCAGCGGTCG GGCTGAACGG |
| | 1501 | GGGGTTCGTG CATACAGCCC AGCTTGGAGC GAACGACCTA CACCGAACTG AGATACCTAC |
| | 1561 | AGCGTGAGCT ATGAGAAAGC GCCACGCTTC CCGAAGGGAG AAAGGCGGAC AGGTATCCGG |
| | 1621 | TAAGCGGCAG GGTCGGAACA GGAGAGCGCA CGAGGGAGCT TCCAGGGGGA AACGCCTGGT |
| | 1681 | ATCTTTATAG TCCTGTCGGG TTTCGCCACC TCTGACTTGA GCGTCGATTT TTGTGATGCT |
| | 1741 | CGTCAGGGGG GCGGAGCCTA TGGAAAAACG CCAGCAACGC GGCCTTTTTA CGGTTCCTGG |
| | 1801 | CCTTTTGCTG GCCTTTTGCT CACATGTTCT TTCCTGCGTT ATCCCCTGAT TCTGTGGATA |
| | 1861 | ACCGTATTAC CGCCTTTGAG TGAGCTGATA CCGCTCGCCG CAGCCGAACG ACCGAGCGCA |
| | 1921 | GCGAGTCAGT GAGCGAGGAA GCGGAAGAGC GCCCAATACG CAAACCGCCT CTCCCCGCGC |
| | 1981 | GTTGGCCGAT TCATTAATGC AGCTGGCACG ACAGGTTTCC CGACTGGAAA GCGGGCAGTG |
| | 2041 | AGCGCAACGC AATTAATGTG AGTTAGCTCA CTCATTAGGC ACCCCAGGCT TTACACTTTA |
| | 2101 | TGCTTCCGGC TCGTATGTTG TGTGGAATTG TGAGCGGATA ACAATTTCAC ACAGGAAACA |
| | 2161 | GCTATGACCA TGATTACGCC AAGCTTTGGA GCCTTTTTTT TGGAGATTTT CAAC |

| | |
|---|---|
| ! | 2215-3021 Hc expression cassette |

| | | |
|---|---|---|
| ! | | Signal sequence------------------------------------------- |
| ! | | 1 2 3 4 5 6 7 8 9 10 11 12 13 14 15 |
| ! | | M K K L L F A I P L V V P F V |
| | 2215 | atg aag aag ctc ctc ttt gct atc ccg ctc gtc gtt cct ttt gtg |
| ! | | |
| ! | | Signal---------------- FR1------------------------------- |
| ! | | 16 17 18 19 20 21 22 23 24 25 26 27 28 29 30 |
| ! | | A Q P A M A E V Q L L E S G G |
| | 2260 | gcc cag ccg gcc atg gcc gaa gtt caa ttg tta gag tct ggt ggc |
| ! | | |
| ! | | FR1------------------------------------------------------- |
| ! | | 31 32 33 34 35 36 37 38 39 40 41 42 43 44 45 |
| ! | | G L V Q P G G S L R L S C A A |
| | 2305 | ggt ctt gtt cag cct ggt ggt tct tta cgt ctt tct tgc gct gct |
| ! | | |
| ! | | FR1------------------- CDR1-------------- FR2----------- |
| ! | | 46 47 48 49 50 51 52 53 54 55 56 57 58 59 60 |
| ! | | S G F T F S S Y A M S W V R Q |
| | 2350 | tcc gga ttc act ttc tct agt tac gct atg tcc tgg gtt cgc caa |
| ! | | |
| ! | | FR2----------------------------------- CDR2-------------- |
| ! | | 61 62 63 64 65 66 67 68 69 70 71 72 73 74 75 |
| ! | | A P G K G L E W V S A I S G S |
| | 2395 | gct cct ggt aaa ggt ttg gag tgg gtt tct gct atc tct ggt tct |
| ! | | |
| ! | | CDR2-------------- FR3----------------------------------- |
| ! | | 76 77 78 79 80 81 82 83 84 85 86 87 88 89 90 |
| ! | | G G S T Y Y A D S V K G R F T |
| | 2440 | ggt ggc agt act tac tat gct gac tcc gtt aaa ggt cgc ttc act |
| ! | | |
| ! | | FR3------------------------------------------------------- |
| ! | | 91 92 93 94 95 96 97 98 99 100 101 102 103 104 105 |
| ! | | I S R D N S K N T L Y L Q M N |
| | 2485 | atc tct aga gac aac tct aag aat act ctc tac ttg cag atg aac |
| ! | | |
| ! | | FR3--------------------------------------------------- CDR3-- |
| ! | | 106 107 108 109 110 111 112 113 114 115 116 117 118 119 120 |
| ! | | S L R A E D T A V Y Y C A R A |
| | 2530 | agc tta agg gct gag gac act gca gtc tac tat tgt gcg aga gcc |
| ! | | |
| ! | | CDR3------------------------------------------------------- |
| ! | | 121 122 123 124 125 126 127 128 129 130 131 132 133 134 135 |
| ! | | S A S N G S A Y A A I A P G L |
| | 2575 | tct gcc tct aat ggt agt gct tac gct gct ata gct cct gga ctt |
| ! | | |
| ! | | CDR3--- FR4------------------------------------------------ |
| ! | | 136 137 138 139 140 141 142 143 144 145 146 147 148 149 150 |
| ! | | D Y W G Q G T L V T V S S A S |
| | 2620 | gac tac tgg ggc cag gga acc ctg gtc acc gtc tca agc gcc tcc |
| ! | | |
| ! | | 151 152 153 154 155 156 157 158 159 160 161 162 163 164 165 |
| ! | | T K G P S V F P L A P S S K S |
| | 2665 | acc aag ggt ccg tcg gtc ttc ccg cta gca ccc tcc tcc aag agc |
| ! | | |
| ! | | 166 167 168 169 170 171 172 173 174 175 176 177 178 179 180 |
| ! | | T S G G T A A L G C L V K D Y |
| | 2710 | acc tct ggg ggc aca gcg gcc ctg ggc tgc ctg gtc aag gac tac |
| ! | | |
| ! | | 181 182 183 184 185 186 187 188 189 190 191 192 193 194 195 |
| ! | | F P E P V T V S W N S G A L T |
| | 2755 | ttc ccc gaa ccg gtg acg gtg tcg tgg aac tca ggc gcc ctg acc |
| ! | | |
| ! | | 196 197 198 199 200 201 202 203 204 205 206 207 208 209 210 |
| ! | | S G V H T F P A V L Q S S G L |
| | 2800 | agc ggc gtc cac acc ttc ccg gct gtc cta cag tct agc gga ctc |
| ! | | |
| ! | | 211 212 213 214 215 216 217 218 219 220 221 222 223 224 225 |
| ! | | Y S L S S V V T V P S S S L G |
| | 2845 | tac tcc ctc agc agc gta gtg acc gtg ccc tct agc agc tta ggc |
| ! | | |
| ! | | 226 227 228 229 230 231 232 233 234 235 236 237 238 239 240 |
| ! | | T Q T Y I C N V N H K P S N T |
| | 2890 | acc cag acc tac atc tgc aac gtg aat cac aag ccc agc aac acc |
| ! | | |
| ! | | 241 242 243 244 245 246 247 248 249 250 251 252 253 254 255 |
| ! | | K V D K K V E P K S C A A A G |
| | 2935 | aag gtg gac aag aaa gtt gag ccc aaa tct tgt gcg gcc gct ggt |
| ! | | |
| ! | | 256 257 258 259 260 261 262 263 264 265 266 267 268 269 |
| ! | | K P I P N P L L G L D S T • |
| | 2980 | aag cct atc cct aac cct ctc ctc ggt ctc gat tct acg tga |
| ! | | |
| | 3022 | TAACTTCAC CGGTCAACGC GTGATGAGAA TTCACTGGCC |
| | 3061 | GTCGTTTTAC AACGTCGTGA CTGGGAAAAC CCTGGCGTTA CCCAACTTAA TCGCCTTGCA |
| | 3121 | GCACATCCCC CTTTCGCCAG CTGGCGTAAT AGCGAAGAGG CCCGCACCGA TCGCCCTTCC |
| | 3181 | CAACAGTTGC GCAGCCTGAA TGGCGAATGG CGCCTGATGC GGTATTTTCT CCTTACGCAT |
| | 3241 | CTGTGCGGTA TTTCACACCG CATACGTCAA AGCAACCATA GTCTCAGTAC AATCTGCTCT |
| | 3301 | GATGCCGCAT AGTTAAGCCA GCCCCGACAC CCGCCAACAC CCGCTGACGC GCCCTGACAG |
| | 3361 | GCTTGTCTGC TCCCGGCATC CGCTTACAGA CAAGCTGTGA CCGTCTCCGG GAGCTGCATG |
| | 3421 | TGTCAGAGGT TTTCACCGTC ATCACCGAAA CGCGCGA |

TABLE 4
DNA Sequence of DY3F63

| | | | | |
|---|---|---|---|---|
| LOCUS | AY754023 | 9030 bp | DNA | circular SYN 10-MAR-2005 |

| | |
|---|---|
| SOURCE | Enterobacteria phage M13 vector DY3F63 |
| | Hogan, S., Rem, L., Frans, N., Daukandt, M., Pieters, H., van |
| | Hegelsom, R., Coolen-van Neer, N., Nastri, H.G., Rondon, I.J., |
| | Leeds, J., Hufton, S.E., Huang, L., Kashin, I., Devlin, M., |
| | Kuang, G., Steukers, M., Viswanathan, M., Nixon, A.E., |
| | Sexton, D.J., Hoogenboom, H.R. and Ladner, R.C. |
| TITLE | Generation of high-affinity human antibodies by combining |
| | donor-derived and synthetic complementarity-determining- |
| | region diversity |
| JOURNAL | Nat. Biotechnol. 23 (3), 344-348 (2005) |
| PUBMED | 15723048 |
| REFERENCE | 2 (bases 1 to 9030) |
| AUTHORS | Ladner, R.C., Hoogenboom, H.R., Hoet, R.M., Cohen, E.H., |
| | Kashin, I., Rondon, I.J., Rem, L., Frans, N., Schoonbroodt, |
| | S., Kent, R.B., Rookey, K. and Hogan, S. |
| TITLE | Direct Submission |
| JOURNAL | Submitted (13-SEP-2004) Research, Dyax Corp, 300 Technology |
| | Square, Cambridge, MA 02139, USA |
| FEATURES | Location/Qualifiers |
| source | 1 . . . 9030 |
| | /organism = “Enterobacteria phage M13 vector DY3F63” |
| | /mol_type = “other DNA” |
| | /db_xref = “taxon:296376” |
| | /note = “derived from M13mp18 phage cloning vector in |
| | GenBank Accession Number M77815; has high-affinity |
| | synthetic and donor-derived diversity” |
| gene | 6145 . . . 7005 |
| | /gene = “bla” |
| CDS | 6145 . . . 7005 |
| | /gene = “bla” |
| | /note = “ApR” |
| | /codon_start = 1 |
| | /transl_table = 11 |
| | /product = “beta-lactamase” |
| | /protein id = “AAV54522.1” |
| | /db_xref = “GI: 55669167” |
| | /translation = “MSIQHFRVALIPFFAAFCLPVFAHPETLVKVKDAEDQLGALVGY |
| | IELDLNSGKILESFRPEERFPMMSTFKVLLCGAVLSRIDAGQEQLGRRIHYSQNDLVE |
| | YSPVTEKHLTDGMTVRELCSAAITMSDNTAANLLLTTIGGPKELTAFLHNMGDHVTRL |
| | DRWEPELNEAIPNDERDTTMPVAMATTLRKLLTGELLTLASRQQLIDWMEADKVAGPL |
| | LRSALPAGWFIADKSGAGERGSRGIIAALGPDGKPSRIVVIYTTGSQATMDERNRQIA |
| | EIGASLIKHW” (SEQ ID NO:31) |
| misc_feature | 7425 . . . 7481 |
| | /note = “encodes light chain signal sequence; antibody |
| | stuffer” |
| misc_feature | 7491 . . . 7536 |
| | /note = “encodes light chain antibody stuffer” |
| misc_feature | 7563 . . . 7628 |
| | /note = “encodes heavy chain signal sequence; antibody |
| | /note = “encodes heavy chain antibody stuffer” |
| | /note = “encodes domain 3 of protein III; antibody stuffer” |
| ORIGIN | |

(SEQ ID NO:32)

| | |
|---|---|
| 1 | aatgctacta ctattagtag aattgatgcc accttttcag ctcgcgcccc aaatgaaaat |
| 61 | atagctaaac aggttattga ccatttgcga aatgtatcta atggtcaaac taaatctact |
| 121 | cgttcgcaga attgggaatc aactgttata tggaatgaaa cttccagaca ccgtacttta |
| 181 | gttgcatatt taaaacatgt tgagctacag cattatattc agcaattaag ctctaagcca |
| 241 | tccgcaaaaa tgacctctta tcaaaaggag caattaaagg tactctctaa tcctgacctg |
| 301 | ttggagtttg cttccggtct ggttcgcttt gaagctcgaa ttaaaacgcg atatttgaag |
| 361 | tctttcgggc ttcctcttaa tctttttgat gcaatccgct ttgcttctga ctataatagt |
| 421 | cagggtaaag acctgatttt tgatttatgg tcattctcgt tttctgaact gtttaaagca |
| 481 | tttgaggggg attcaatgaa tatttatgac gattccgcag tattggacgc tatccagtct |
| 541 | aaacatttta ctattacccc ctctggcaaa acttcttttg caaaagcctc tcgctatttt |
| 601 | ggtttttatc gtcgtctggt aaacgagggt tatgatagtg ttgctcttac tatgcctcgt |
| 661 | aattcctttt ggcgttatgt atctgcatta gttgaatgtg gtattcctaa atctcaactg |
| 721 | atgaatcttt ctacctgtaa taatgttgtt ccgttagttc gttttattaa cgtagatttt |
| 781 | tcttcccaac gtcctgactg gtataatgag ccagttctta aaatcgcata aggtaattca |
| 841 | caatgattaa agttgaaatt aaaccatctc aagcccaatt tactactcgt tctggtgttt |
| 901 | ctcgtcaggg caagccttat tcactgaatg agcagctttg ttacgttgat ttgggtaatg |
| 961 | aatatccggt tcttgtcaag attactcttg atgaaggtca gccagcctat gcgcctggtc |
| 1021 | tgtacaccgt tcatctgtcc tctttcaaag ttggtcagtt cggttccctt atgattgacc |
| 1081 | gtctgcgcct cgttccggct aagtaacatg gagcaggtcg cggatttcga cacaatttat |
| 1141 | caggcgatga tacaaatctc cgttgtactt tgtttcgcgc ttggtataat cgctgggggt |
| 1201 | caaagatgag tgttttagtg tattcttttg cctctttcgt tttaggttgg tgccttcgta |
| 1261 | gtggcattac gtattttacc cgtttaatgg aaacttcctc atgaaaaagt ctttagtcct |
| 1321 | caaagcctct gtagccgttg ctaccctcgt tccgatgctg tctttcgctg ctgagggtga |
| 1381 | cgatcccgca aaagcggcct ttaactccct gcaagcctca gcgaccgaat atatcggtta |
| 1441 | tgcgtgggcg atggttgttg tcattgtcgg cgcaactatc ggtatcaagc tgtttaagaa |
| 1501 | attcacctcg aaagcaagct gataaaccga tacaattaaa ggctcctttt ggagcctttt |
| 1561 | tttttggaga ttttcaacgt gaaaaaatta ttattcgcaa ttcctttagt tgttcctttc |
| 1621 | tattctcact ccgctgaaac tgttgaaagt tgtttagcaa aatcccatac agaaaattca |
| 1681 | tttactaacg tctggaaaga cgacaaaact ttagatcgtt acgctaacta tgagggctgt |
| 1741 | ctgtggaatg ctacaggcgt tgtagtttgt actggtgacg aaactcagtg ttacggtaca |
| 1801 | tgggttccta ttgggcttgc tatccctgaa aatgagggtg gtggctctga gggtggcggt |
| 1861 | tctgagggtg gcggttctga gggtggcggt actaaacctc ctgagtacgg tgatacacct |
| 1921 | attccgggct atacttatat caaccctctc gacggcactt atccgcctgg tactgagcaa |
| 1981 | aaccccgcta atcctaatcc ttctcttgag gagtctcagc ctcttaatac tttcatgttt |
| 2041 | cagaataata ggttccgaaa taggcagggg gcattaactg tttatacggg cactgttact |
| 2101 | caaggcactg accccgttaa aacttattac cagtacactc ctgtatcatc aaaagccatg |
| 2161 | tatgacgctt actggaacgg taaattcaga gactgcgctt tccattctgg ctttaatgag |
| 2221 | gatttatttg tttgtgaata tcaaggccaa tcgtctgacc tgcctcaacc tcctgtcaat |
| 2281 | gctggcggcg gctctggtgg tggttctggt ggcggctctg agggtggtgg ctctgagggt |
| 2341 | ggcggttctg agggtggcgg ctctgaggga ggcggttccg gtggtggctc tggttccggt |
| 2401 | gattttgatt atgaaaagat ggcaaacgct aataaggggg ctatgaccga aaatgccgat |
| 2461 | gaaaacgcgc tacagtctga cgctaaaggc aaacttgatt ctgtcgctac tgattacggt |
| 2521 | gctgctatcg atggtttcat tggtgacgtt tccggccttg ctaatggtaa tggtgctact |
| 2581 | ggtgattttg ctggctctaa ttcccaaatg gctcaagtcg gtgacggtga taattcacct |
| 2641 | ttaatgaata atttccgtca atatttacct tccctccctc aatcggttga atgtcgccct |
| 2701 | tttgtctttg gcgctggtaa accatatgaa ttttctattg attgtgacaa aataaactta |
| 2761 | ttccgtggtg tctttgcgtt tcttttatat gttgccacct ttatgtatgt attttctacg |
| 2821 | tttgctaaca tactgcgtaa taaggagtct taatcatgcc agttcttttg ggtattccgt |
| 2881 | tattattgcg tttcctcggt ttccttctgg taactttgtt cggctatctg cttacttttc |
| 2941 | ttaaaaaggg cttcggtaag atagctattg ctatttcatt gtttcttgct cttattattg |
| 3001 | ggcttaactc aattcttgtg ggttatctct ctgatattag cgctcaatta ccctctgact |
| 3061 | ttgttcaggg tgttcagtta attctcccgt ctaatgcgct tccctgtttt tatgttattc |
| 3121 | tctctgtaaa ggctgctatt ttcatttttg acgttaaaca aaaaatcgtt tcttatttgg |
| 3181 | attgggataa ataatatggc tgtttatttt gtaactggca aattaggctc tggaaagacg |
| 3241 | ctcgttagcg ttggtaagat tcaggataaa attgtagctg ggtgcaaaat agcaactaat |
| 3301 | cttgatttaa ggcttcaaaa cctcccgcaa gtcgggaggt tcgctaaaac gcctcgcgtt |
| 3361 | cttagaatac cggataagcc ttctatatct gatttgcttg ctattgggcg cggtaatgat |
| 3421 | tcctacgatg aaaataaaaa cggcttgctt gttctcgatg agtgcggtac ttggtttaat |
| 3481 | acccgttctt ggaatgataa ggaaagacag ccgattattg attggtttct acatgctcgt |
| 3541 | aaattaggat gggatattat ttttcttgtt caggacttat ctattgttga taaacaggcg |
| 3601 | cgttctgcat tagctgaaca tgttgtttat tgtcgtcgtc tggacagaat tactttacct |
| 3661 | tttgtcggta ctttatattc tcttattact ggctcgaaaa tgcctctgcc taaattacat |
| 3721 | gttggcgttg ttaaatatgg cgattctcaa ttaagcccta ctgttgagcg ttggctttat |
| 3781 | actggtaaga atttgtataa cgcatatgat actaaacagg ctttttctag taattatgat |
| 3841 | tccggtgttt attcttattt aacgccttat ttatcacacg gtcggtattt caaaccatta |
| 3901 | aatttaggtc agaagatgaa attaactaaa atatatttga aaaagttttc tcgcgttctt |
| 3961 | tgtcttgcga ttggatttgc atcagcattt acatatagtt atataaccca acctaagccg |
| 4021 | gaggttaaaa aggtagtctc tcagacctat gattttgata aattcactat tgactcttct |
| 4081 | cagcgtctta atctaagcta tcgctatgtt ttcaaggatt ctaagggaaa attaattaat |
| 4141 | agcgacgatt tacagaagca aggttattca ctcacatata ttgatttatg tactgtttcc |
| 4201 | attaaaaaag gtaattcaaa tgaaattgtt aaatgtaatt aattttgttt tcttgatgtt |
| 4261 | tgtttcatca tcttcttttg ctcaggtaat tgaaatgaat aattcgcctc tgcgcgattt |
| 4321 | tgtaacttgg tattcaaagc aatcaggcga atccgttatt gtttctcccg atgtaaaagg |
| 4381 | tactgttact gtatattcat ctgacgttaa acctgaaaat ctacgcaatt tctttatttc |
| 4441 | tgttttacgt gcaaataatt ttgatatggt aggttctaac ccttccataa ttcagaagta |
| 4501 | taatccaaac aatcaggatt atattgatga attgccatca tctgataatc aggaatatga |
| 4561 | tgataattcc gctccttctg gtggtttctt tgttccgcaa aatgataatg ttactcaaac |
| 4621 | ttttaaaatt aataacgttc gggcaaagga tttaatacga gttgtcgaat tgtttgtaaa |
| 4681 | gtctaatact tctaaatcct caaatgtatt atctattgac ggctctaatc tattagttgt |
| 4741 | tagtgctcct aaagatattt tagataacct tcctcaattc ctttcaactg ttgatttgcc |
| 4801 | aactgaccag atattgattg agggtttgat atttgaggtt cagcaaggtg atgctttaga |
| 4861 | tttttcattt gctgctggct ctcagcgtgg cactgttgca ggcggtgtta atactgaccg |
| 4921 | cctcacctct gttttatctt ctgctggtgg ttcgttcggt atttttaatg gcgatgtttt |
| 4981 | agggctatca gttcgcgcat taaagactaa tagccattca aaaatattgt ctgtgccacg |
| 5041 | tattcttacg ctttcaggtc agaagggttc tatctctgtt ggccagaatg tcccttttat |
| 5101 | tactggtcgt gtgactggtg aatctgccaa tgtaaataat ccatttcaga cgattgagcg |
| 5161 | tcaaaatgta ggtatttcca tgagcgtttt tcctgttgca atggctggcg gtaatattgt |
| 5221 | tctggatatt accagcaagg ccgatagttt gagttcttct actcaggcaa gtgatgttat |
| 5281 | tactaatcaa agaagtattg ctacaacggt taatttgcgt gatggacaga ctcttttact |
| 5341 | cggtggcctc actgattata aaaacacttc tcaggattct ggcgtaccgt tcctgtctaa |
| 5401 | aatcccttta atcggcctcc tgtttagctc ccgctctgat tctaacgagg aaagcacgtt |
| 5461 | atacgtgctc gtcaaagcaa ccatagtacg cgccctgtag cggcgcatta agcgcggcgg |
| 5521 | gtgtggtggt tacgcgcagc gtgaccgcta cacttgccag cgccctagcg cccgctcctt |
| 5581 | tcgctttctt cccttccttt ctcgccacgt tcgccggctt tccccgtcaa gctctaaatc |
| 5641 | gggggctccc tttagggttc cgatttagtg ctttacggca cctcgacccc aaaaaacttg |
| 5701 | atttgggtga tggttcacgt agtgggccat cgccctgata gacggttttt cgccctttga |
| 5761 | cgttggagtc cacgttcttt aatagtggac tcttgttcca aactggaaca acactcaacc |
| 5821 | ctatctcggg ctattctttt gatttataag ggattttgcc gatttcggaa ccaccatcaa |
| 5881 | acaggatttt cgcctgctgg ggcaaaccag cgtggaccgc ttgctgcaac tctctcaggg |
| 5941 | ccaggcggtg aagggcaatc agctgttgcc cgtctcactg gtgaaaagaa aaaccaccct |
| 6001 | ggatccaagc ttgcaggtgg cacttttcgg ggaaatgtgc gcggaacccc tatttgttta |
| 6061 | tttttctaaa tacattcaaa tatgtatccg ctcatgagac aataaccctg ataaatgctt |
| 6121 | caataatatt gaaaaaggaa gagtatgagt attcaacatt tccgtgtcgc ccttattccc |
| 6181 | ttttttgcgg cattttgcct tcctgttttt gctcacccag aaacgctggt gaaagtaaaa |
| 6241 | gatgctgaag atcagttggg cgcactagtg ggttacatcg aactggatct caacagcggt |
| 6301 | aagatccttg agagttttcg ccccgaagaa cgttttccaa tgatgagcac ttttaaagtt |
| 6361 | ctgctatgtg gcgcggtatt atcccgtatt gacgccgggc aagagcaact cggtcgccgc |
| 6421 | atacactatt ctcagaatga cttggttgag tactcaccag tcacagaaaa gcatcttacg |
| 6481 | gatggcatga cagtaagaga attatgcagt gctgccataa ccatgagtga taacactgcg |
| 6541 | gccaacttac ttctgacaac gatcggagga ccgaaggagc taaccgcttt tttgcacaac |
| 6601 | atgggggatc atgtaactcg ccttgatcgt tgggaaccgg agctgaatga agccatacca |
| 6661 | aacgacgagc gtgacaccac gatgcctgta gcaatggcaa caacgttgcg caaactatta |
| 6721 | actggcgaac tacttactct agcttcccgg caacaattaa tagactggat ggaggcggat |
| 6781 | aaagttgcag gaccacttct gcgctcggcc cttccggctg gctggtttat tgctgataaa |
| 6841 | tctggagccg gtgagcgtgg gtctcgcggt atcattgcag cactggggcc agatggtaag |
| 6901 | ccctcccgta tcgtagttat ctacacgacg gggagtcagg caactatgga tgaacgaaat |
| 6961 | agacagatcg ctgagatagg tgcctcactg attaagcatt ggtaactgtc agaccaagtt |
| 7021 | tactcatata tactttagat tgatttaaaa cttcattttt aatttaaaag gatctaggtg |
| 7081 | aagatccttt ttgataatct catgaccaaa atcccttaac gtgagttttc gttccactgt |
| 7141 | acgtaagacc cccaagcttg tcgactgaat ggcgaatggc gctttgcctg gtttccggca |
| 7201 | ccagaagcgg tgccggaaag ctggctggag tgcgatcttc ctgacgctcg agcgcaacgc |
| 7261 | aattaatgtg agttagctca ctcattaggc accccaggct ttacacttta tgcttccggc |
| 7321 | tcgtatgttg tgtggaattg tgagcggata acaatttcac acaggaaaca gctatgacca |
| 7381 | tgattacgcc aagctttgga gccttttttt tggagatttt caacgtgaaa aaattattat |
| 7441 | tcgcaattcc tttagttgtt cctttctatt ctcacagtgc acagtgatag actagttaga |
| 7501 | cgcgtgctta aaggcctcca atcctcttgg cgcgccaatt ctatttcaag gagacagtca |
| 7561 | taatgaaata cctattgcct acggcagccg ctggattgtt attactcgcg gcccagccgg |
| 7621 | ccctctgata agatatcact tgtttaaact ctgcttggcc ctcttggcct tctagtagac |
| 7681 | ttgcggccgc acatcatcat caccatcacg gggccgcaga acaaaaactc atctcagaag |
| 7741 | aggatctgaa tggggccgca gaggctagct ctgctagtgg cgacttcgac tacgagaaaa |
| 7801 | tggctaatgc caacaaaggc gccatgactg agaacgctga cgagaatgct ttgcaaagcg |
| 7861 | atgccaaggg taagttagac agcgtcgcga ccgactatgg cgccgccatc gacggcttta |
| 7921 | tcggcgatgt cagtggtttg gccaacggca acggagccac cggagacttc gcaggttcga |
| 7981 | attctcagat ggcccaggtt ggagatgggg acaacagtcc gcttatgaac aactttagac |
| 8041 | agtaccttcc gtctcttccg cagagtgtcg agtgccgtcc attcgttttc ggtgccggca |
| 8101 | agccttacga gttcagcatc gactgcgata agatcaatct tttccgcggc gttttcgctt |
| 8161 | tcttgctata cgtcgctact ttcatgtacg ttttcagcac tttcgccaat attttacgca |
| 8221 | acaaagaaag ctagtgatct cctaggaagc ccgcctaatg agcgggcttt ttttttctgg |
| 8281 | tatgcatcct gaggccgata ctgtcgtcgt cccctcaaac tggcagatgc acggttacga |
| 8341 | tgcgcccatc tacaccaacg tgacctatcc cattacggtc aatccgccgt ttgttcccac |
| 8401 | ggagaatccg acgggttgtt actcgctcac atttaatgtt gatgaaagct ggctacagga |
| 8461 | aggccagacg cgaattattt ttgatggcgt tcctattggt taaaaaatga gctgatttaa |
| 8521 | caaaaattta atgcgaattt taacaaaata ttaacgttta caatttaaat atttgcttat |
| 8581 | acaatcttcc tgtttttggg gcttttctga ttatcaaccg gggtacatat gattgacatg |
| 8641 | ctagttttac gattaccgtt catcgattct cttgtttgct ccagactctc aggcaatgac |
| 8701 | ctgatagcct ttgtagatct ctcaaaaata gctaccctct ccggcattaa tttatcagct |
| 8761 | agaacggttg aatatcatat tgatggtgat ttgactgtct ccggcctttc tcaccctttt |
| 8821 | gaatctttac ctacacatta ctcaggcatt gcatttaaaa tatatgaggg ttctaaaaat |
| 8881 | ttttatcctt gcgttgaaat aaaggcttct cccgcaaaag tattacaggg tcataatgtt |
| 8941 | tttggtacaa ccgatttagc tttatgctct gaggctttat tgcttaattt tgctaattct |
| 9001 | ttgccttgcc tgtatgattt attggatgtt |
| // | |

TABLE 5
DNA sequence of pMJD21 (5957 bp)

(SEQ ID NO:33)

| | |
|---|---|
| 1 | gacgaaaggg cctcgtgata cgcctatttt tataggttaa tgtcatgata ataatggttt |
| 61 | cttagacgtc aggtggcact tttcggggaa atgtgcgcgg aacccctatt tgtttatttt |
| 121 | tctaaataca ttcaaatatg tatccgctca tgagacaata accctgataa atgcttcaat |
| 181 | aatattgaaa aaggaagagt atgagtattc aacatttccg tgtcgccctt attccctttt |
| 241 | ttgcggcatt ttgccttcct gtttttgctc acccagaaac gctggtgaaa gtaaaagatg |
| 301 | ctgaagatca gttgggtgcc cgagtgggtt acatcgaact ggatctcaac agcggtaaga |
| 361 | tccttgagag ttttcgcccc gaagaacgtt ttccaatgat gagcactttt aaagttctgc |
| 421 | tatgtggcgc ggtattatcc cgtattgacg ccgggcaaga gcaactcggt cgccgcatac |
| 481 | actattctca gaatgacttg gttgagtact caccagtcac agaaaagcat cttacggatg |
| 541 | gcatgacagt aagagaatta tgcagtgctg ccataaccat gagtgataac actgcggcca |
| 601 | acttacttct gacaacgatc ggaggaccga aggagctaac cgcttttttg cacaacatgg |
| 661 | gggatcatgt aactcgcctt gatcgttggg aaccggagct gaatgaagcc ataccaaacg |
| 721 | acgagcgtga caccacgatg cctgtagcaa tggcaacaac gttgcgcaaa ctattaactg |
| 781 | gcgaactact tactctagct tcccggcaac aattaataga ctggatggag gcggataaag |
| 841 | ttgcaggacc acttctgcgc tcggcccttc cggctggctg gtttattgct gataaatctg |
| 901 | gagccggtga gcgtgggtct cgcggtatca ttgcagcact ggggccagat ggtaagccct |
| 961 | cccgtatcgt agttatctac acgacgggga gtcaggcaac tatggatgaa cgaaatagac |
| 1021 | agatcgctga gataggtgcc tcactgatta agcattggta actgtcagac caagtttact |
| 1081 | catatatact ttagattgat ttaaaacttc atttttaatt taaaaggatc taggtgaaga |
| 1141 | tcctttttga taatctcatg accaaaatcc cttaacgtga gttttcgttc cactgagcgt |
| 1201 | cagaccccgt agaaaagatc aaaggatctt cttgagatcc tttttttctg cgcgtaatct |
| 1261 | gctgcttgca aacaaaaaaa ccaccgctac cagcggtggt ttgtttgccg gatcaagagc |
| 1321 | taccaactct ttttccgaag gtaactggct tcagcagagc gcagatacca aatactgttc |
| 1381 | ttctagtgta gccgtagtta ggccaccact tcaagaactc tgtagcaccg cctacatacc |
| 1441 | tcgctctgct aatcctgtta ccagtggctg ctgccagtgg cgataagtcg tgtcttaccg |
| 1501 | ggttggactc aagacgatag ttaccggata aggcgcagcg gtcgggctga acggggggtt |
| 1561 | cgtgcataca gcccagcttg gagcgaacga cctacaccga actgagatac ctacagcgtg |
| 1621 | agctatgaga aagcgccacg cttcccgaag ggagaaaggc ggacaggtat ccggtaagcg |
| 1681 | gcagggtcgg aacaggagag cgcacgaggg agcttccagg gggaaacgcc tggtatcttt |
| 1741 | atagtcctgt cgggtttcgc cacctctgac ttgagcgtcg atttttgtga tgctcgtcag |
| 1801 | gggggcggag cctatggaaa aacgccagca acgcggcctt tttacggttc ctggcctttt |
| 1861 | gctggccttt tgctcacatg ttctttcctg cgttatcccc tgattctgtg gataaccgta |
| 1921 | ttaccgcctt tgagtgagct gataccgctc gccgcagccg aacgaccgag cgcagcgagt |
| 1981 | cagtgagcga ggaagcggaa gagcgcccaa tacgcaaacc gcctctcccc gcgcgttggc |
| 2041 | cgattcatta atgcagctgg cacgacaggt ttcccgactg gaaagcgggc agtgagcgca |
| 2101 | acgcaattaa tgtgagttag ctcactcatt aggcacccca ggctttacac tttatgcttc |
| 2161 | cggctcgtat gttgtgtgga attgtgagcg gataacaatt tcacacagga aacagctatg |
| 2221 | accatgatta cgccaagctt tggagccttt tttttggaga ttttcaacgt gaaaaaatta |
| 2281 | ttattcgcaa ttcctttagt tgttcctttc tattctcaca gtgcacaggt ccaactgcag |
| 2341 | gagctcgaga tcaaacgtgg aactgtggct gcaccatctg tcttcatctt cccgccatct |
| 2401 | gatgagcagt tgaaatctgg aactgcctct gttgtgtgcc tgctgaataa cttctatccc |
| 2461 | agagaggcca aagtacagtg gaaggtggat aacgccctcc aatcgggtaa ctcccaggag |
| 2521 | agtgtcacag agcaggacag caaggacagc acctacagcc tcagcagcac cctgacgctg |
| 2581 | agcaaagcag actacgagaa acacaaagtc tacgcctgcg aagtcaccca tcagggcctg |
| 2641 | agttcaccgg tgacaaagag cttcaacagg ggagagtgtt aataaggcgc gcctaaccat |
| 2701 | ctatttcaag gaacagtctt aatgaaaaag cttttattca tgatcccgtt agttgtaccg |
| 2761 | ttcgtggccc agccggcctc tgctgaagtt caattgttag agtctggtgg cggtcttgtt |
| 2821 | cagcctggtg gttctttacg tctttcttgc gctgcttccg gagcttcaga tctgtttgcc |
| 2881 | tttttgtggg gtggtgcaga tcgcgttacg gagatcgacc gactgcttga gcaaaagcca |
| 2941 | cgcttaactg ctgatcaggc atgggatgtt attcgccaaa ccagtcgtca ggatcttaac |
| 3001 | ctgaggcttt ttttacctac tctgcaagca gcgacatctg gtttgacaca gagcgatccg |
| 3061 | cgtcgtcagt tggtagaaac attaacacgt tgggatggca tcaatttgct taatgatgat |
| 3121 | ggtaaaacct ggcagcagcc aggctctgcc atcctgaacg tttggctgac cagtatgttg |
| 3181 | aagcgtaccg tagtggctgc cgtacctatg ccatttgata agtggtacag cgccagtggc |
| 3241 | tacgaaacaa cccaggacgg cccaactggt tcgctgaata taagtgttgg agcaaaaatt |
| 3301 | ttgtatgagg cggtgcaggg agacaaatca ccaatcccac aggcggttga tctgtttgct |
| 3361 | gggaaaccac agcaggaggt tgtgttggct gcgctggaag atacctggga gactctttcc |
| 3421 | aaacgctatg gcaataatgt gagtaactgg aaaacaccgg caatggcctt aacgttccgg |
| 3481 | gcaaataatt tctttggtgt accgcaggcc gcagcggaag aaacgcgtca tcaggcggag |
| 3541 | tatcaaaacc gtggaacaga aaacgatatg attgttttct caccaacgac aagcgatcgt |
| 3601 | cctgtgcttg cctgggatgt ggtcgcaccc ggtcagagtg ggtttattgc tcccgatgga |
| 3661 | acagttgata agcactatga agatcagctg aaaatgtacg aaaattttgg ccgtaagtcg |
| 3721 | ctctggttaa cgaagcagga tgtggaggcg cataaggagt tctagagaca actctaagaa |
| 3781 | tactctctac ttgcagatga acagcttaag tctgagcatt cggtccgggc aacattctcc |
| 3841 | aaactgacca gacgacacaa acggcttacg ctaaatcccg cgcatgggat ggtaaagagg |
| 3901 | tggcgtcttt gctggcctgg actcatcaga tgaaggccaa aaattggcag gagtggacac |
| 3961 | agcaggcagc gaaacaagca ctgaccatca actggtacta tgctgatgta aacggcaata |
| 4021 | ttggttatgt tcatactggt gcttatccag atcgtcaatc aggccatgat ccgcgattac |
| 4081 | ccgttcctgg tacgggaaaa tgggactgga aagggctatt gccttttgaa atgaacccta |
| 4141 | aggtgtataa cccccagcag ctagccatat tctctcggtc accgtctcaa gcgcctccac |
| 4201 | caagggccca tcggtcttcc cgctagcacc ctcctccaag agcacctctg ggggcacagc |
| 4261 | ggccctgggc tgcctggtca aggactactt ccccgaaccg gtgacggtgt cgtggaactc |
| 4321 | aggcgccctg accagcggcg tccacacctt cccggctgtc ctacagtcta gcggactcta |
| 4381 | ctccctcagc agcgtagtga ccgtgccctc ttctagcttg ggcacccaga cctacatctg |
| 4441 | caacgtgaat cacaagccca gcaacaccaa ggtggacaag aaagttgagc ccaaatcttg |
| 4501 | tgcggccgca catcatcatc accatcacgg ggccgcagaa caaaaactca tctcagaaga |
| 4561 | ggatctgaat ggggccgcag aggctagttc tgctagtaac gcgtcttccg gtgattttga |
| 4621 | ttatgaaaag atggcaaacg ctaataaggg ggctatgacc gaaaatgccg atgaaaacgc |
| 4681 | gctacagtct gacgctaaag gcaaacttga ttctgtcgct actgattacg gtgctgctat |
| 4741 | cgatggtttc attggtgacg tttccggcct tgctaatggt aatggtgcta ctggtgattt |
| 4801 | tgctggctct aattcccaaa tggctcaagt cggtgacggt gataattcac ctttaatgaa |
| 4861 | taatttccgt caatatttac cttccctccc tcaatcggtt gaatgtcgcc cttttgtctt |
| 4921 | tggcgctggt aaaccatatg aattttctat tgattgtgac aaaataaact tattccgtgg |
| 4981 | tgtctttgcg tttcttttat atgttgccac ctttatgtat gtattttcta cgtttgctaa |
| 5041 | catactgcgt aataaggagt cttaatgaaa cgcgtgatga gaattcactg gccgtcgttt |
| 5101 | tacaacgtcg tgactgggaa aaccctggcg ttacccaact taatcgcctt gcagcacatc |
| 5161 | cccctttcgc cagctggcgt aatagcgaag aggcccgcac cgatcgccct tcccaacagt |
| 5221 | tgcgcagcct gaatggcgaa tggcgcctga tgcggtattt tctccttacg catctgtgcg |
| 5281 | gtatttcaca ccgcatacgt caaagcaacc atagtacgcg ccctgtagcg gcgcattaag |
| 5341 | cgcggcgggt gtggtggtta cgcgcagcgt gaccgctaca cttgccagcg ccttagcgcc |
| 5401 | cgctcctttc gctttcttcc cttcctttct cgccacgttc gccggctttc cccgtcaagc |
| 5461 | tctaaatcgg gggctccctt tagggttccg atttagtgct ttacggcacc tcgaccccaa |
| 5521 | aaaacttgat ttgggtgatg gttcacgtag tgggccatcg ccctgataga cggtttttcg |
| 5581 | ccctttgacg ttggagtcca cgttctttaa tagtggactc ttgttccaaa ctggaacaac |
| 5641 | actcaactct atctcgggct attcttttga tttataaggg attttgccga tttcggtcta |
| 5701 | ttggttaaaa aatgagctga tttaacaaaa atttaacgcg aattttaaca aaatattaac |
| 5761 | gtttacaatt ttatggtgca gtctcagtac aatctgctct gatgccgcat agttaagcca |
| 5821 | gccccgacac ccgccaacac ccgctgacgc gccctgacgg gcttgtctgc tcccggcatc |
| 5881 | cgcttacaga caagctgtga ccgtctccgg gagctgcatg tgtcagaggt tttcaccgtc |
| 5941 | atcaccgaaa cgcgcga |

### REFERENCES

The contents of all cited references including literature references, issued patents, published or non-published patent applications cited throughout this application as well as those listed below are hereby expressly incorporated by reference in their entireties. In case of conflict, the present application, including any definitions herein, will control.

Hoet, R. M. et al. Generation of high-affinity human antibodies by combining donor-derived and synthetic complementarity-determining-region diversity. Nat Biotechnol 23, 344-348 (2005).

Lu, D. et al. Tailoring in vitro selection for a picomolar affinity human antibody directed against vascular endothelial growth factor receptor 2 for enhanced neutralizing activity. J Biol Chem 278, 43496-43507 (2003).

### EQUIVALENTS

A number of embodiments of the invention have been described. Nevertheless, it will be understood that various modifications may be made without departing from the spirit and scope of the invention. Accordingly, other embodiments are within the scope of the following claims.

We claim:

1. A method of producing specific binding pair (SBP) members with affinity for a predetermined target, wherein the SBP comprises a first polypeptide chain and a second polypeptide chain, which method comprises:
(i) providing host cells that comprise a first population of vectors comprising a population of genetic material encoding one or more of the first polypeptide chains which have been selected to have one or more desirable properties, wherein the first polypeptide chains are secreted from the host cells;
(ii) infecting the cells with a second population of vectors that comprises a diverse population of genetic material that encodes the second polypeptide chains, wherein the second polypeptide chain is fused to a component of a secreted replicable genetic display package (RGDP) for display of the second polypeptide chains at the surface of RGDPs;
(iii) expressing the first and second polypeptide chains within the host cells to form a library of SBP members displayed at the surface of the RGDPs, wherein the first and second polypeptide chains are associated at the surface of the RGDPs; and
(iv) selecting SBP members for binding to the predetermined target.

2. The method of claim 1, wherein the first polypeptide chains comprise antibody heavy chains (HC) or antigen binding fragments thereof.

3. The method of claim 1, wherein the second polypeptide chains comprise antibody light chains (LC) or antigen binding fragments thereof.

4. The method of claim 1, wherein the first polypeptide chains comprise antibody light chains (LC) or antigen binding fragments thereof.

5. The method of claim 1, wherein the second polypeptide chains comprise antibody heavy chains (HC) or antigen binding fragments thereof.

6. The method of claim 1, wherein the first vectors are plasmids.

7. The method of claim 1, wherein the first vectors are phage vectors.

8. The method of claim 1, wherein the second vectors are phage vectors.

9. The method of claim 1, wherein the first population of vectors encodes 1 to 1000 different first polypeptide chains.

10. The method of claim 1, wherein the second vectors encode a genetically diverse population of 105 or more different second polypeptide chains.

11. The method of claim 1, wherein the selecting comprises an ELISA (Enzyme-Linked ImmunoSorbent Assay).

12. The method of claim 1 further comprising isolating specific binding pair members that bind to the predetermined target.

13. The method of claim 1 further comprising infecting a fresh sample of host cells of step (i) with the selected RGDPs from step (iv).

14. The method of claim 1, wherein the first population is divided into two or more subpopulations and phage produced from one subpopulation are selected and propagated separately from phage produced in other populations.

15. A method of producing specific binding pair (SBP) members with improved affinity for a predetermined target, wherein the SBP comprises a first polypeptide chain and a second polypeptide chain, which method comprises:
introducing into host cells:
(i) a first population of vectors comprising nucleic acid encoding one or more of the first polypeptide chains which have been selected to have affinity for the predetermined target fused to a component of a secreted replicable genetic display package (RGDP) for display of the polypeptide chains at the surface of RGDPs; and
(ii) a second population of vectors comprising nucleic acid encoding a genetically diverse population of the second polypeptide chain;
the first vectors being packaged in infectious RGDPs and their introduction into host cells being by infection into host cells harboring the second vectors; or
the second vectors being packaged in infectious RGDPs and their introducing into host cells being by infection into host cells harboring the first vectors;
expressing the first and second polypeptide chains within the host cells to form a library of the SBP members displayed by RGDPs, at least one of the populations being expressed from nucleic acid that is capable of being packaged using the RGDP component, whereby the genetic materials of each the RGDP encodes a polypeptide chain of the SBP member displayed at its surface; and
selecting members of the population for high-affinity binding to the predetermined target.

16. The method of claim 15, wherein the first population is divided into two or more subpopulations and phage produced from one subpopulation are selected and propagated separately from phage produced in other populations.

17. The method of claim 1, wherein the first population of vectors encodes 1000 or fewer first polypeptide chains.

18. The method of claim 1, wherein the first population of vectors encodes 100 or fewer first polypeptide chains.

19. The method of claim 1, wherein the first population of vectors encodes 20 or fewer first polypeptide chains.

20. The method of claim 1, wherein the first population of vectors encodes 10 or fewer first polypeptide chains.

21. The method of claim 1, wherein the first population of vectors encodes 1 first polypeptide chain.
